(19) United States
(12) Patent Application Publication
(10) Pub. No.: US 2015/0254433 A1
(43) Pub. Date: Sep. 10, 2015

(54) Methods and Models for Determining Likelihood of Cancer Drug Treatment Success Utilizing Predictor Biomarkers, and Methods of Diagnosing and Treating Cancer Using the Biomarkers

(71) Applicants: Bruce MACHER, San Mateo, CA (US); Leslie TIMPE, Burlingame, CA (US); Ten-Yang YEN, Pleasanton, CA (US); Alexandra PIRYATINSKA, San Carlos, CA (US)

(72) Inventors: Bruce MACHER, San Mateo, CA (US); Leslie TIMPE, Burlingame, CA (US); Ten-Yang YEN, Pleasanton, CA (US); Alexandra PIRYATINSKA, San Carlos, CA (US)

(21) Appl. No.: 14/558,618

(22) Filed: Dec. 2, 2014

Related U.S. Application Data
(60) Provisional application No. 61/948,501, filed on Mar. 5, 2014.

(51) Int. Cl. G06F 19/00; A61K 31/52; A61K 31/7064; A61K 31/475; A61K 31/436; A61K 31/337

(52) U.S. Cl. CPC G06F 19/706; A61K 31/436; A61K 31/337; A61K 31/7064; A61K 31/475; A61K 31/52; G06F 19/704

(57) ABSTRACT

A method of identifying one or more biomarkers associated with one or more drugs effective to stop or repress proliferation of cancer cells, and a system for predicting effectiveness of the same. The method includes statistically analyzing (i) a first dataset of expression levels of proteins or glycoproteins in the cancer cells and (ii) a second dataset of responses of the cancer cells to drugs to identify at least one biomarker associated with effective repression of the cancer cells, and correlating or associating at least one protein or glycoprotein biomarker with a response of the cells to at least one of the drugs effective to stop or repress the proliferation of the cancer cells. The protein and/or glycoprotein expression level datasets may be generated experimentally or taken from published information. The method advantageously determines and/or predicts drug sensitivity of various cancer cells using protein and glycoprotein biomarkers.

### RELATED APPLICATIONS

This application claims the benefit of U.S. Provisional Patent Application No. 61/948,501, filed Mar. 5, 2014, which is incorporated herein by reference in its entirety.

### FIELD OF THE INVENTION

The present invention generally relates to the fields of identifying and using predictive biomarkers for diagnosing and treating cancer. More specifically, embodiments of the present invention pertain to methods of identifying and using biomarkers associated with one or more drugs that stop or repress proliferation of cancer cells, and systems for conducting and/or implementing the same. The present invention provides an efficient method of determining and/or predicting sensitivity of various cancer cells and/or cancer to particular drugs using protein and/or glycoprotein biomarkers.

### DISCUSSION OF THE BACKGROUND

Cancer is a group of diseases characterized by uncontrolled growth and spread of abnormal cells, which can result in death. Cancer is caused by both external factors (e.g., tobacco, chemicals, radiation, and infectious organisms) and internal factors (e.g., inherited mutations, hormones, immune conditions and mutations). These factors may act together or in sequence to initiate or promote the development of cancer cells. Cancer cell lines are cells that are subcultured under certain conditions in a laboratory. Generally, cancer cell lines are used in research to study the biology of cancer and to test cancer treatments.

Chemotherapy is the use of an anticancer drug to treat cancerous cells. Chemotherapy has been used for many years and is one of the most common treatments for cancer. In most cases, chemotherapy works by interfering with the cancer cell's ability to divide. Different groups of drugs work differently to fight cancer cells. Chemotherapy may be used alone for some types of cancer, and in combination with other treatments such as radiation or surgery for other types of cancer. Often, a combination of chemotherapy drugs is used to fight a specific cancer. Certain chemotherapy drugs may be given in a specific order depending on the type of cancer being treated.

Typically, breast cancer is the most prevalent of all cancers in American women. Breast cancer is a relatively complex disease with respect to the type of tumor, chance of recurrence, and responsiveness to therapy and/or treatment. Also, the complexity of breast cancer includes variety in protein expression found in tumors.

Biomarkers provide information about a particular tumor, and can be used to monitor the recurrence of cancer. Biomarkers used in cancer diagnosis and/or treatment include glycoproteins that consist of extracellular and secreted proteins, for example prostate serum antigen or prostate-specific antigen (prostate cancer), carcinoembryonic antigen (colorectal cancer), and CA125 (ovarian cancer).

The U.S. Food and Drug Administration has approved several dozen drugs for breast cancer treatment, but only a few predictive biomarkers are available to guide their use. The exceptions are compounds that interfere with estrogen receptor (ER) signaling, for which the levels of estrogen or progesterone receptor (PR) are predictive, especially for response to hormone therapy. In addition, the over-expression of human epidermal growth factor receptor 2 (HER2) predicts sensitivity to pertuzumab, trastuzumab and lapatinib. Generally, the rate of approval of new biomarkers is low, and fell between 1994 and 2005 (Ludwig and Weinstein, Nature Reviews Cancer 5:845-856, 2005). Thus, additional biomarkers for identifying tumors that are sensitive to drugs already approved for use in breast cancer or in clinical development would be significantly valuable.

Recently, large datasets describing the effects of various drugs on the growth of cancer cells in culture have been generated for the purpose of accelerating the preclinical evaluation of new compounds. One of largest datasets with respect to the number of drugs and breast cancer cell lines describes the effects of ninety (90) different drugs on seventy (70) different breast cancer cell lines. The dataset includes measurements of the concentration of each drug that causes a 50% reduction in the proliferation of cells in culture (i.e., GI₅₀). According to the dataset, the sensitivity to various drugs in cell lines varies significantly, sometimes by more than four orders of magnitude. Acquired resistance to chemotherapeutics or targeted agents is recognized and is being studied intensively. The variation in sensitivities to the ninety drugs displayed by the cell lines in culture is probably not due to resistance acquired from previous exposure to these drugs. There appears to be a relatively large amount of intrinsic variability in the responses to drugs by these tumor-derived cell lines. These intrinsic differences in sensitivity, if replicated in breast tumors, could explain some of the variability in responses of tumors to chemotherapeutic drugs or targeted agents.

In the past, several efforts to identify biomarkers that predict drug response in breast cancer using mRNA signatures have been attempted. Typically, the signatures include a large number of mRNA's. For example, a 74 gene model was constructed to predict complete pathologic response to paclitaxel, fluorouracil, doxorubicin and cyclophosphamide. Response to docetaxel was predicted with a set of 85 mRNA's. Seventy-nine genes were used to predict survival after treatment with doxorubicin. Another example of using mRNA signatures includes a 32 gene signature that predicts the persistence of malignancy after neoadjuvant liposomal doxorubicin/paclitaxel therapy. Conventionally, the mRNA was derived from a tumor or sections of a tumor, rather than cell lines, making the interpretation difficult due to the variety of cell types present in the tumor tissue. The large number of genes in the various signatures may reflect the small amount of signal in mRNA as compared to protein.

More recently, there have been efforts to solve the problem of predicting the responses of cancer cell lines to drugs. Predictor data includes gene mutation, copy number variation, methylation and gene expression data, protein data, and receptor signaling networks. Other conventional methods that have been used in attempt to solve the prediction problem of drug effectiveness on cell lines include machine learning and statistical methods.

Several related statistical methods have been employed recently in modeling drug response in cancer cell lines for both mRNA and protein data. Ridge regression has been used as part of an effort to predict patient drug response based on the drug responses of cancer cell lines. Ridge regression applies a different penalty than lasso regression, and provides a regression model with more predictors. If a regression problem has p predictor variables, the ridge penalty forces all, or most, of the corresponding regression coefficients to small values, but not to zero. Hence, the number of predictors in the final model is still p. Ridge regression can give models with low prediction error, but may not eliminate any predictors.

Elastic net regression has also been used recently for predicting drug response. In two cases the predictor variables are proteins, measured by mass spectrometry. Elastic net regression combines the penalties of lasso and ridge regression. The result is often a model with many predictors, but fewer than the maximum possible number, p. Elastic net regression can also give models with low prediction error. However, a need is felt for a method of identifying drug(s) effective to stop or repress proliferation of cancer cells using a smaller number of predictor biomarkers (e.g., a few accurate and effective predictor biomarker(s), such as 1-3 biomarkers).

### SUMMARY OF THE INVENTION

Embodiments of the present invention relate to a method of identifying one or more of a plurality of drugs effective to stop or repress proliferation of cancer cells using protein and/or glycoprotein biomarkers, and a system for predicting effectiveness of the drug(s) using the biomarkers. The method generally includes statistically analyzing (i) a first dataset of expression levels of a plurality of proteins or glycoproteins in the cancer cells and (ii) a second dataset of responses of the cancer cells to a plurality of drugs to identify one or more biomarkers associated with effective repression of the cancer cells, and correlating or associating at least one of the one or more biomarkers with a response of the cancer cells to at least one of the plurality of drugs effective to stop or repress the proliferation of the cancer cells. In exemplary embodiments of the present invention, the first dataset comprises expression levels of glycoproteins, and the biomarker(s) are glycoprotein biomarker(s). Alternatively, the first dataset comprises expression levels of proteins, and the biomarkers are protein biomarker(s). In some examples, the biomarker consists of a single glycoprotein biomarker. Alternatively, the present method identifies two or three protein or glycoprotein biomarkers associated with a drug effective to stop or repress proliferation of the cancer cells. The first and second datasets may be statistically analyzed by a regression analysis (e.g., lasso regression). Preferably, each drug that targets a particular target has a unique biomarker or set of biomarkers.

In various embodiments of the present invention, the cancer cells may be breast cancer cells, lung cancer cells, melanoma cells, prostate cancer cells, ovarian cancer cells, bladder cancer cells, endometrial cancer cells, kidney cancer cells, pancreatic cancer cells, colorectal cancer cells, lymphoma cells, CNS cancer cells, thyroid cancer cells, or leukemia cells. However, the present invention may be particularly applicable to identifying biomarkers form breast cancer cells and/or lung cancer cells.

In further embodiments, the drug(s) target epidermal growth factor receptor (EGFR) and/or human epidermal growth factor receptor 2 (HER2), microtubules and/or tubulin, nucleic acids (e.g., DNA), mammalian targets of rapamycin (mTORs), alpha serine/threonine-protein kinase (AKT1), phosphatidylinositol-3-kinase (PI3′ kinase), or cyclin-dependent kinase (CDK), and the biomarker(s) may be receptor tyrosine-protein kinase erbB-2 (PO4626), cathepsin B (P07858), cadherin-13 (P55290), bone marrow stromal antigen 2 (Q10589), neprilysin (P08473), large neutral amino acids transporter small subunit 1 (Q01650), integrin alpha-6 (P23229), dipeptidyl peptidase 1 (P53634), collagen alpha-1 (VI) chain (P12109), neutral amino acid transporter B (Q15758), transcobalamin-1 (P20061), sushi domain-containing protein 2 (Q9UGT4), podocalyxin (000592), laminin subunit beta-1 (P07942), dipeptidyl peptidase 1 (P53634), gamma-interferon-inducible lysosomal thiol reductase (P13284), neuroplalstin (Q9Y639), CD44 antigen (P16070), ubiquitin carboxyl-terminal hydrolase 5 (P45974), solute carrier family 2, facilitated glucose transporter membrane 1 (P11166), and alpha-aminoadipic semialdehyde dehydrogenase (P49419), CD276 antigen (Q5ZPR3), cathepsin Z (Q9UBR2), and serpin H1 (P50454); lysosome membrane protein 2 (Q14108), alpha-aminoadipic semialdehyde dehydrogenase (P49419), isochorismatase domain-containing protein 1 (Q96CN7), beta-mannosidase (000462), glucose-6-phosphate 1-dehydrogenase (P11413), ribonuclease UK114 (P52758), tropomyosin alpha-4 chain (P67936), ganglioside GM2 activator (P17900), granulins (P28799), steryl-sulfatase (P08842), insulin-like growth factor-binding protein 7 (Q16270), lysosomal pro-x carboxypeptidase (P42785), receptor tyrosine-protein kinase erbB-2 (PO4626), transmembrane emp24 domain-containing protein 7 (Q9Y3B3), arylsulfatase A (P15289), mucin-1 (P15941), G2/mitotic-specific cyclin-B1 (P14635), G1/S-specific cyclin-E1 (P24864), thioredoxin-dependent peroxide reductase, mitochondrial (P30048), acylaminoacyl-peptidase, putative (ApeH-1; Q97YB2), and/or importin subunit alpha-1 (P52292).

In more specific embodiments of the present invention, the drug that targets epidermal growth factor receptor (EGFR) and human epidermal growth factor receptor 2 (HER2) comprises (i) afatinib, and the glycoprotein biomarker(s) include one or more of receptor tyrosine-protein kinase erbB-2 (PO4626), cathepsin B (P07858), cadherin-13 (P55290), bone marrow stromal antigen 2 (Q10589), and sushi domain-containing protein 2 (Q9UGT4), (ii) erlotinib, and the glycoprotein biomarker(s) include one or more of sushi domain-containing protein 2 (Q9UGT4), neprilysin (P08473), large neutral amino acids transporter small subunit 1 (Q01650), integrin alpha-6 (P23229), dipeptidyl peptidase 1 (P53634), collagen alpha-1 (VI) chain (P12109), and neutral amino acid transporter B (Q15758), (iii) gefitinib, and the glycoprotein biomarker(s) include one or more of transcobalamin-1 (P20061), sushi domain-containing protein 2 (Q9UGT4), podocalyxin (000592), large neutral amino acids transporter small subunit 1 (Q01650), laminin subunit beta-1 (P07942), and dipeptidyl peptidase 1 (P53634), and (iv) lapatinib, and the glycoprotein biomarker(s) include one or more of receptor tyrosine-protein kinase erbB-2 (PO4626), gamma-interferon-inducible lysosomal thiol reductase (P13284), neuroplalstin (Q9Y639), cathepsin B (P07858), CD44 antigen (P16070), and bone marrow stromal antigen 2 (Q10589).

In other more specific embodiments of the present invention, the drug that targets microtubules comprises (i) paclitaxel, and the biomarker(s) may include one or more proteins selected from ubiquitin carboxyl-terminal hydrolase 5 (P45974), solute carrier family 2, facilitated glucose transporter membrane 1 (P11166), and alpha-aminoadipic semialdehyde dehydrogenase (P49419), or the biomarker may include one or more glycoproteins selected from CD276 antigen (Q5ZPR3), cathepsin Z (Q9UBR2), and serpin H1 (P50454); and/or (ii) docetaxel, and the biomarker(s) may include at least one protein selected from lysosome membrane protein 2 (Q14108), alpha-aminoadipic semialdehyde dehydrogenase (P49419), and isochorismatase domain-containing protein 1 (Q96CN7), or at least one glycoprotein selected from beta-mannosidase (000462), cathepsin Z (Q9UBR2), and serpin H1 (P50454).

In further more specific embodiments of the present invention, the drug that targets tubulin comprises vinorelbine, and the biomarker(s) may include one or more of glucose-6-phosphate 1-dehydrogenase (P11413), ribonuclease UK114 (P52758), and tropomyosin alpha-4 chain (P67936); and the drug that targets nucleic acids (e.g., DNA) comprises gemcitabine, and the glycoprotein biomarker(s) include one or more of ganglioside GM2 activator (P17900), granulins (P28799), and steryl-sulfatase (P08842).

In even further embodiments of the present invention, the drug that targets mTOR inhibitors comprises (i) everolimus, and the glycoprotein biomarker(s) include one or more of insulin-like growth factor-binding protein 7 (Q16270), lysosomal pro-x carboxypeptidase (P42785), and receptor tyrosine-protein kinase erbB-2 (PO4626), and/or (ii) temsirolimus, and the glycoprotein biomarker includes one or more of transmembrane emp24 domain-containing protein 7 (Q9Y3B3), arylsulfatase A (P15289), and receptor tyrosine-protein kinase erbB-2 (PO4626).

In other more specific embodiments of the present invention, the drug that targets PI3′ kinase inhibitor comprises BEZ235, and the glycoprotein biomarker(s) include one or more of collagen alpha-1 (VI) chain (P12109), large neutral amino acids transporter small subunit 1 (Q01650), mucin-1 (P15941), and receptor tyrosine-protein kinase erbB-2 (PO4626).

In additional more specific embodiments of the present invention, the drug that targets CDK inhibitors is palbociclib, and biomarker(s) include one or more proteins selected from G2/mitotic-specific cyclin-B1 (P14635), G1/S-specific cyclin-E1 (P24864), thioredoxin-dependent peroxide reductase, mitochondrial (P30048), acylaminoacyl-peptidase, putative (ApeH-1; Q97YB2), and importin subunit alpha-1 (P52292).

The present invention further relates to a method of treating cancer, generally comprising identifying and quantifying at least one protein or glycoprotein biomarker in cancer cells from a patient, identifying one or more of a plurality of drugs that effectively stop or repress proliferation of the cancer cells from a correlation or association of the protein or glycoprotein biomarker(s) with effectiveness of the drugs, and administering the drug(s) in a pharmaceutically acceptable carrier or excipient to the patient having the cancer cells in an amount effective to stop or repress the proliferation of the cancer cells. The biomarker(s) may include one or more glycoprotein biomarkers.

In various embodiments, the drug(s) may be administered orally, intravenously, or by chemotherapy infusion. For example, the effective drug may be administered orally via a pill or a liquid formulation comprising a dose of the drug in an amount effective to stop proliferation of the cancer cells, in a pharmaceutically acceptable carrier or excipient. The drug may be administered intravenously or by chemotherapy infusion via an intravenous (IV) bag, an IV drip, or a syringe containing a dose of effective drug in an amount effective to stop proliferation of the cancer cells, in a pharmaceutically acceptable aqueous carrier or excipient. The method may further include administering an additional cancer therapy selected from radiation therapy, surgery, and a combination thereof to the patient.

Further embodiments of the present invention relate to a system configured to predict effectiveness of one or more of a plurality of drugs to stop or repress proliferation of cancer cells. The system generally comprises a memory storing (i) a first dataset including expression levels of a plurality of proteins or glycoproteins in a plurality of cancer cell lines, and (ii) a second dataset including an effectiveness of each of the plurality of drugs to stop or repress proliferation of the cancer cell lines; and a computer configured to statistically analyze the first and second datasets to (i) identify and/or select at least one biomarker for each of the cancer cell lines and (ii) correlate or associate at least one of the plurality of drugs that effectively stops or represses proliferation of the cells in at least one of the cancer cell lines with the biomarker(s) for each of the cancer cell lines. The computer may be configured to statistically analyze the first and second datasets using lasso regression. In addition, the first dataset may include expression levels of glycoproteins, and the biomarker(s) may include one or more glycoprotein biomarkers. In such embodiments, the system may further include a third dataset that includes expression levels of a plurality of proteins in the same or a different plurality of cancer cell lines, in which case the biomarker(s) may include or further include one or more protein biomarkers associated with the drug(s) effective to stop or repress proliferation cancer cells. In the various embodiments, the effectiveness of each drug to stop or repress proliferation of the cancer cell line is determined by a response that measures a concentration of the drug(s) that causes 50% reduction in proliferation of cancer cells.

The present invention advantageously identifies protein and/or glycoprotein biomarkers for cancer cell sensitivity to a relatively large number of drugs, quantitatively based on the expression level of 1-3 protein or glycoprotein biomarkers. These and other advantages of the present invention will become readily apparent from the detailed description of various embodiments below.

### BRIEF DESCRIPTION OF THE DRAWINGS

FIG. 1 shows a table indicating the probability that HER2 (glycoprotein and MRM datasets) or HER2p1248 (RPPA data) occurred in a lasso regression model for five EGFR/HER2 inhibiting drugs according to examples of the present invention.

FIG. 2 shows the relation between HER2 expression and the drug sensitivity for five EGFR or HER2 inhibiting drugs according to one or more examples of the present invention.

FIGS. 3A-3B shows fitted lapatinib sensitivities using two or three protein predictors according to one or more examples of the present invention.

FIG. 4 shows fitting of AKT1 inhibitors with AKTp478 and PDK1.

FIGS. 5A-5B shows frequency distributions of multiple coefficient of correlations R² for single predictor models (FIG. 5A) and three-predictor models (FIG. 5B).

FIG. 6 shows a table of twelve single predictor models for three protein or glycoprotein datasets according to examples of the present invention.

FIG. 7 shows an association between glycoprotein expression levels and the corresponding mRNA, measured for 184 glycoprotein/mRNA pairs in 19 cell lines.

FIG. 8A shows a graph of the root MSPE, and FIG. 8B shows a graph of the root MSE, in which both are proportional to the range of drug sensitivities, the range for a drug being the difference in sensitivities between the most sensitive and least sensitive cell lines studied.

FIGS. 9A-9C show estimates of prediction error relative to mean square error for various models with one predictor, according to examples of the present invention.

FIGS. 10A-10C show modeling of mTOR inhibitors according to one or more examples of the present invention.

FIGS. 11A-11D show modeling of taxanes (e.g., paclitaxel and docetaxel), gemcitabine, and vinorelbine according to examples of the present invention.

### DETAILED DESCRIPTION

Reference will now be made in detail to various embodiments of the invention, examples of which are illustrated in the accompanying drawings. While the invention will be described in conjunction with certain embodiments and examples, it will be understood that they are not intended to limit the invention. On the contrary, the invention is intended to cover alternatives, modifications and equivalents that may be included within the spirit and scope of the invention as defined by the appended claims. Furthermore, in the following detailed description of the present invention, numerous specific details are set forth in order to provide a thorough understanding of the present invention. However, it will be readily apparent to one skilled in the art that the present invention may be practiced without these specific details. In other instances, well-known methods, procedures, components, and circuits have not been described in detail so as not to unnecessarily obscure aspects of the present invention.

The present invention concerns a method of identifying one or more of a plurality of drugs effective to stop or repress proliferation of cancer cells. The method generally includes statistically analyzing (i) a first dataset of expression levels of a plurality of proteins or glycoproteins in the cancer cells and (ii) a second dataset of responses of the cancer cells to a plurality of drugs to identify one or more biomarkers (e.g., one or more protein biomarkers or glycoprotein biomarkers) associated with effective repression of the cancer cells, and from the biomarker(s) and the responses, correlating or associating at least one of the one or more biomarkers with a response of the cancer cells to at least one of the drugs effective to stop or repress the proliferation of the cancer cells.

In a further aspect, the present invention concerns a method of treating cancer. The method generally comprises identifying at least one biomarker (e.g., one or more protein or glycoprotein biomarkers) in cancer cells from a patient, identifying one or more of a plurality of drugs that effectively stop or repress proliferation of the cancer cells from a correlation or association of the biomarker(s) with effectiveness of the drug(s) to stop or repress the proliferation of the cancer cells, and administering the correlated or associated drug(s) in a pharmaceutically acceptable carrier or excipient to the patient having the cancer cells in an amount effective to stop or repress the proliferation of the cancer cells.

In yet a further aspect, the present invention concerns a system configured to predict effectiveness of one or more drugs to stop or repress proliferation of cancer cells. The system generally comprises a memory storing (i) a first dataset including expression levels of a plurality of proteins or glycoproteins in the plurality of the cancer cell lines, and (ii) a second dataset including an effectiveness of each of a plurality of drugs to stop or repress proliferation of the cancer cell lines, and a computer configured to statistically analyze the first and second datasets to (i) identify and/or select at least one protein or glycoprotein biomarker for each of the cancer cell lines and (ii) correlate or associate at least one of the drugs that effectively stops or represses proliferation of the cancer cells in each of the cancer cell lines with the biomarker(s) for each of the cancer cell lines. The invention, in its various aspects, will be explained in greater detail below with regard to exemplary embodiments.

Exemplary Methods and Models for Correlating or Associating Biomarkers with Drugs Effective to Stop or Repress Proliferation of Cancer Cells

The present invention concerns a method of identifying one or more drugs effective to stop or repress proliferation or growth of cancer cells. The method includes statistically analyzing (i) a dataset of expression levels of proteins or glycoproteins in cancer cells (e.g., one or more cancer cell lines) and (ii) a dataset of responses of the cancer cells (or cancer cell lines) to various drugs to identify at least one biomarker associated with effective repression of the cancer cells using one or more of the drugs. In addition, the method includes correlating or associating the biomarker(s) with a response of the cancer cells to at least one of the drugs that effectively stops or represses the growth of the cancer cells. The present invention advantageously provides a method of identifying biomarkers (e.g., protein and/or glycoprotein biomarkers) that accurately and effectively determine which drug or drugs may be effective in stopping or repressing proliferation of cancer cells. Thus, the present invention provides an effective, efficient, and practical method for determining drug effectiveness on various cancer cells and for diagnosing and treating patients with cancer using the protein and/or glycoprotein biomarkers and the drug(s) associated and/or correlated with the biomarkers.

The datasets that are statistically analyzed may include protein and/or glycoprotein expression levels in one or more (e.g., a plurality of) cancer cell lines. The datasets may include one or more glycoprotein datasets and/or one or more protein datasets. The dataset(s) may be generated experimentally or be publicly available. The drug response profiles and the protein or glycoprotein expression level datasets may all be quantitative. In exemplary embodiments of the present disclosure, an indirect dataset includes expression levels of proteins in a plurality of (e.g., 5 or more, 10 or more, 20 or more, etc.) cancer cell lines, or separately, expression levels of glycoproteins in a plurality of (e.g., 5, 10, 20, or more) cancer cell lines. Expression levels of proteins and/or glycoproteins in various cancer cell lines can be determined using various methods, including but not limited to, mass spectrometry (e.g., using a multiple reaction monitoring [MRM] assay), reverse phase protein array (RPPA) analysis, or immunoblotting (e.g., Western blotting).

Another dataset that is statistically analyzed using lasso regression (e.g., a least absolute shrinkage and selection operator [i.e., lasso] regression model) includes responses of the cancer cell lines to various cancer treatment drugs (e.g., 5, 10, 20, 30, 40, 50 or more drugs). In one example described herein, the datasets include responses from approximately 90 drugs on 70 different breast cancer cell lines. As mentioned elsewhere herein, the drug response data are determined by measuring the concentration of drugs or compounds that causes a 50% reduction (e.g., GI₅₀ or IC₅₀ data) in the proliferation of the cells in culture.

In one example discussed in detail herein, a dataset based on multiple reaction monitoring (MRM) assays for proteins includes 325 proteins in 30 breast cancer cell lines, of which 27 cell lines overlapped with the example drug response dataset disclosed herein. Another example dataset based on RPPA assays for proteins includes 70 cell lines selected based on cancer data, of which 47 cell lines overlapped with the example drug response dataset disclosed herein. An example glycoprotein dataset includes 185 glycoproteins from 22 cell lines that overlap with the cell lines in the example drug response dataset.

The information provided in the protein or glycoprotein and response (i.e., GI₅₀) datasets may come from various cancer cell lines. For example, the cancer cells lines may include, but are not limited to, breast cancer cell lines, lung cancer cell lines, melanoma cell lines, prostate cancer cell lines, ovarian cancer cell lines, bladder cancer cell lines, endometrial cancer cell lines, kidney cancer cell lines, pancreatic cancer cell lines, colorectal cancer cell lines, lymphoma cell lines, CNS cancer cell lines, thyroid cancer cell lines, or leukemia cell lines.

The present method statistically analyzes the database(s) by modeling quantitative drug response data as a function of a number of quantitative predictor variables. In general, the statistical analysis comprises a regression analysis. Regression models can be made when the drug data and the protein or glycoprotein data derive from the same cell lines. The number of common cell lines varies among various publicly available datasets, but is always less than the number of proteins or glycoproteins within the dataset (i.e., the number of possible predictor variables). In such a case, there can be no unique solution to the regression problem for a given drug. However, lasso regression analysis can reduce the number of predictor variables to a relatively small number (e.g., the 1-5 most important). The present approach advantageously uses lasso regression for each drug to identify candidate predictor variables (i.e., biomarkers). Validation of the results in tumor samples enables identifying patients whose tumor(s) will respond to a particular drug, and spares those patients whose tumor(s) can be predicted to be resistant to a drug from the often toxic side effects of the drug.

Generally, in the statistical analysis, the measured effects of a drug on cell proliferation is the response variable. The expression levels of the proteins or glycoproteins are the predictor variables. In cases in which the response is modeled successfully, the predictors become candidate biomarkers for that drug. Actual biomarkers may be selected on the basis of one or more further criteria, such as its expression level relative to other glycoproteins, quantitative expression level reproducibility, ease in isolating and/or identifying the biomarker in the lab or in a diagnostic assay, etc.

Exemplary Statistical Analyses

Least absolute shrinkage and selection operator (i.e., lasso) regression is a form of penalized least squares regression analysis and statistics, in which the size of the penalty is set by a tuning parameter 2, in which 2 is greater than or equal to zero. Lasso regression minimizes the sum of the squared residuals, subject to the constraint that the sum of the absolute values of the regression coefficients is less than a constant, t, which is related to 2. In lasso regression, the statistical model has the following form:

μ=β₀+β₁xᵢ₁+ . . . +βᵢxᵢⱼ (1)

where i=1 . . . n, j=1 . . . p, and μ is the expected value of the response variable, given data. To fit the lasso regression model, the following constraint on the parameters is enforced, as shown by the following equation:

Σⱼ₌₁^{p}|βj★≦t (2)

Lasso regression analysis was used in various examples disclosed herein to create a list of possible biomarkers from a plurality of glycoprotein and protein expression level databases for each of a plurality of drugs for variable selection. The biomarkers from the protein datasets included one or more protein biomarkers, and the biomarkers from the glycoprotein dataset included one or more glycoprotein biomarkers. The biomarkers consisted of a single biomarker, two or three biomarkers, or more than three biomarkers (but generally not more than five biomarkers). To fit a lasso regression, the R software package glmnet or GLMNET (Comprehensive R Archive Network at http://cran.rproject.org/web/packages/glmnet/index.html) was used. The software package performs leave-one-out cross-validation to find the optimal λ.

Due to the constraint imposed by t, some of the regression coefficients βⱼ become zero. Thus, the lasso regression analysis performs a variable selection. As λ increases, the number of nonzero regression coefficients decreases. The optimal λ is chosen using cross-validation. In some cases, the system or model that corresponds to the optimal λ has no predictors. In the exemplary glycoprotein dataset disclosed herein, lasso regression returned at least one predictor for 87 of 90 drugs. Thus, statistically analyzed by lasso regression is a useful and reliable technique for identifying a reasonable number (e.g., 1-5) biomarkers from a given protein or glycoprotein expression level dataset for a reasonable number (e.g., 10-100) of drugs and cancer cell lines (e.g., ten or more), each expressing a relatively large number (e.g., five or more) of proteins or glycoproteins.

Including more biomarkers may advantageously improve the systems or models. However, adding too many biomarkers (e.g., overfitting) may produce a spurious or unworkable system or model that may fit both noise and signal in the data, thus rendering the model less than completely effective on successive data. Based on statistical theory, as the number of predictors (e.g., biomarkers) approaches the number of cell lines (n), the fit of the model to the observed results may improve until a saturated model is reached and the fit is perfect. When too many predictors or biomarkers are used in a model, the measurements and signal(s) may be noisy. Such a model will perform poorly on data other than the set used to devise the model. To avoid overfitting, the example regression models discussed herein were generated from no more than three glycoproteins. When many models with one or more glycoproteins were possible, the Leaps and Bounds algorithm (Furnival, G M and Wilson, R W J; Regressions by leaps and bounds; Technometrics 2000; 42:69-79) was used to find the best model.

Statistical models and/or systems (e.g., regression models) can be constructed for the response profiles of cell lines to a plurality of drugs (e.g., as many as are available in a database) to describe the intrinsic variation in drug sensitivities. In one example, respective profiles to ninety (90) drugs were analyzed. The independent or predictor variables in this example were derived from one glycoprotein dataset and two protein datasets. For most drugs, a quantitative prediction of the cell lines' responses that is statistically significant may be made with three or fewer predictor proteins or glycoproteins. These proteins or glycoproteins are candidate biomarkers for association and/or correlation with a given drug.

In the analyzed example, the three datasets studied included one glycoprotein dataset generated experimentally, and two protein datasets that are publicly available. For the glycoprotein database generated experimentally, the relative levels of the glycoprotein expression were measured by spectral counts obtained via tandem mass spectrometry. Thus, the present statistical analysis may explain or correlate quantitative response data as a function of a number of quantitative predictor variables.

Different datasets may describe or identify different proteins or groups, and different methods may be used to measure the expression levels of the proteins or glycoproteins. Glycoproteins, for example, can be collected from cancer cells (in one example, from breast cancer cell lines) by oxidation of glycans using periodate. After cell lysis and enrichment for glycoproteins, followed by proteolytic digestion, the samples (e.g., tryptic peptides) may be subjected to liquid chromatography to separate and/or isolate the peptides derived from the glycoproteins, and then tandem mass spectrometry may be used to identify the glycoproteins. In one example, the glycoprotein dataset includes 185 glycoproteins from 22 cell lines, in which relative quantitation was achieved by counting identified mass spectra. Many, if not most, glycoproteins are secreted proteins. Other glycoproteins are included in extracellular domains. Thus, glycoprotein datasets may be enriched for proteins that mediate contacts between cells, as well as components of the basement membrane and extracellular matrix. Many proteins and glycoproteins are expressed at different levels in malignant cell lines, as compared to non-malignant cell lines, with a net loss of glycoprotein expression in malignant cell lines.

The expression data in the example(s) disclosed herein were from various breast cancer cell lines that may be classified as luminal, basal, claudin-low, ER positive, or HER2 overexpressing. Breast cancer cell lines also may be of ductal or lobular origin. With respect to these variables (or similar variables in other cancer types), the sets of breast cancer cell lines analyzed herein represent a sufficiently broad spectrum of cell types for statistically meaningful results in breast cancer tumors. Expression level data taken from cell lines from a majority or substantially all of the variable tissue and/or cell types in other types of cancers can represent a variety or spectrum of cancer cells of that type of cancer that is sufficient for statistically meaningful results in other tumors.

In one example, a publicly available protein dataset is a reverse phase protein array (RPPA) dataset, which depends on antibody binding for quantitation. The 70 proteins measured in this dataset were pre-selected on the basis of known linkage to cancer. They include proteins important in the control of cell proliferation, the cell cycle, and DNA repair.

In another example, a publicly available dataset is based on multiple reaction monitoring (MRM) assays for proteins, and expression level data was obtained using mass spectrometry. The dataset includes 325 proteins in 30 breast cancer cell lines, of which 27 overlapped with the drug response dataset used in the examples disclosed herein for effectiveness in the repression of breast cancer cell lines. The proteins were selected for differential expression across the cell lines. They are found in many cellular compartments and contribute to a wide range of biological processes. Quantitation was achieved by comparing the mass spectrum signal intensity to that of a heavy, stable isotope-labelled, reference peptide. Only two proteins, HER2 and cadherin E, are common to all three of the example protein and glycoprotein datasets discussed in detail herein. Other datasets having the same or similar characteristics can be used in the present method.

Successive runs of lasso regression (e.g., using the glmnet or GLMNET package in the R programming language) sometimes give different results. To evaluate the stability of the predictors, the lasso runs were repeated 1000 times, and for each predictor, the number of successes was scored (e.g., by resampling). To fit the lasso regression, a cyclical coordinate descent optimization method may be implemented. Different selections of lambda will give different selections of the variables. To find the optimal lambda, cross validation may be used as described herein. Due to arguably random initial conditions, different runs of the algorithm may lead to slightly different optimal lambdas and/or to different selected variables. The outcomes may vary widely with the drug being studied, but useful results can generally be obtained with reasonable confidence. In some cases, several (e.g., 2-5) predictors can be found in all runs. In other cases (e.g., up to 50% of the time), no predictor is selected. However, using 1000 lasso runs can lead to a relatively large total number of predictors identified for a given drug. However, use of a smaller number of runs (e.g., 5-500, or any number or range of numbers therein) may lead to a smaller number and smaller variability in the biomarkers identification with reasonable confidence in the results.

The responses of various breast cancer cell lines to over 80 drugs were modeled quantitatively using protein or glycoprotein expression data collected by mass spectrometry or reverse phase protein array (RPPA). Statistically significant regression models were created using 1-3 predictor proteins or glycoproteins that fit the observed drug sensitivities of the cancer cell lines to 86 of the 90 drugs modeled or sampled, including (i) drugs currently in use for breast cancer treatment, such as paclitaxel, everolimus, gemcitabine and vinorelbine, and (ii) drugs in development, such as palbociclib or the PI3′ kinase inhibitors (e.g., BEZ235 and GSK2126458). This demonstrates that the present method is reliable and broadly applicable to a wide variety of anti-cancer drugs as cancer cell lines.

Responses to the targeted agents may be modeled by their nominal targets. Many of the drugs studied inhibit specific protein targets, including the epidermal growth factor receptor (EGFR), HER2 (a constitutively active variant of EGFR), AKT kinase (AKT1), mTOR inhibitors, PI3′ kinase inhibitors, and CDK inhibitors. Lasso regression analysis identifies the drug targets when the targets are present in the protein datasets, and the targets effectively model the drug response. For example, lasso regression identified the expected target of several targeted agents when those proteins were in the dataset analyzed, such as HER2 and EGFR for five inhibitors of the EGF receptor, and AKT for two AKT inhibitor drugs.

Examples of specific drugs associated or correlated with one or more protein or glycoprotein biomarkers, where the drugs are classified by their chemical and/or biological targets, are provided as follows.

Predictive Biomarkers for HER2 and/or EGFR Inhibitors

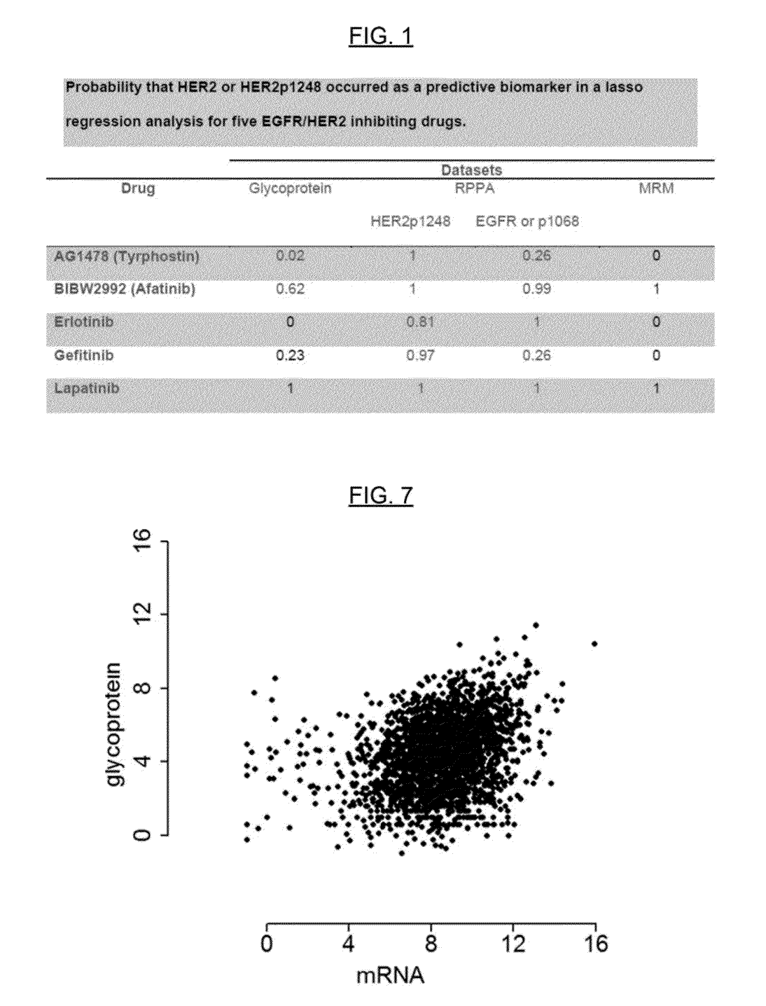

As a control experiment, the probability that HER2 (a glycoprotein) or HER2p1248 (a phosphorylated form of HER2) would be identified as a biomarker in a lasso regression analysis for five EGFR-inhibiting or HER2-inhibiting drugs was determined. The results are shown in the table in FIG. 1. Using the example glycoprotein and the two example protein datasets described herein, candidate protein or glycoprotein biomarkers were selected by lasso regression analysis, which successfully identified human epidermal growth factor receptor 2 (HER2) and epidermal growth factor receptor (EGFR) as predictors for five drugs that are known to be effective in stopping or repressing proliferation of cancer cells. For HER2, the exemplary glycoprotein and MRM datasets were analyzed. For HER2p1248 and EGFR, the exemplary RPPA dataset was analyzed.

Inhibitors targeting EGFR or HER2 include the drugs AG1478, afatinib (BIBW2992), erlotinib, gefitinib and lapatinib. Lapatinib is considered to be a blocker of HER2. Afatinib is in clinical trials for HER2-overexpressing breast cancer. Gefitinib is a targeted agent developed against the receptor for epidermal growth factor (EGFR), which has been approved for treating a subset of patients with lung cancer. For each of these drugs, HER2 or HER2p1248 was identified as a predictive biomarker by lasso regression in at least one dataset. For example, HER2 was found in all lasso analysis for lapatinib in the glycoprotein dataset, but was not identified as a predictor for erlotinib.

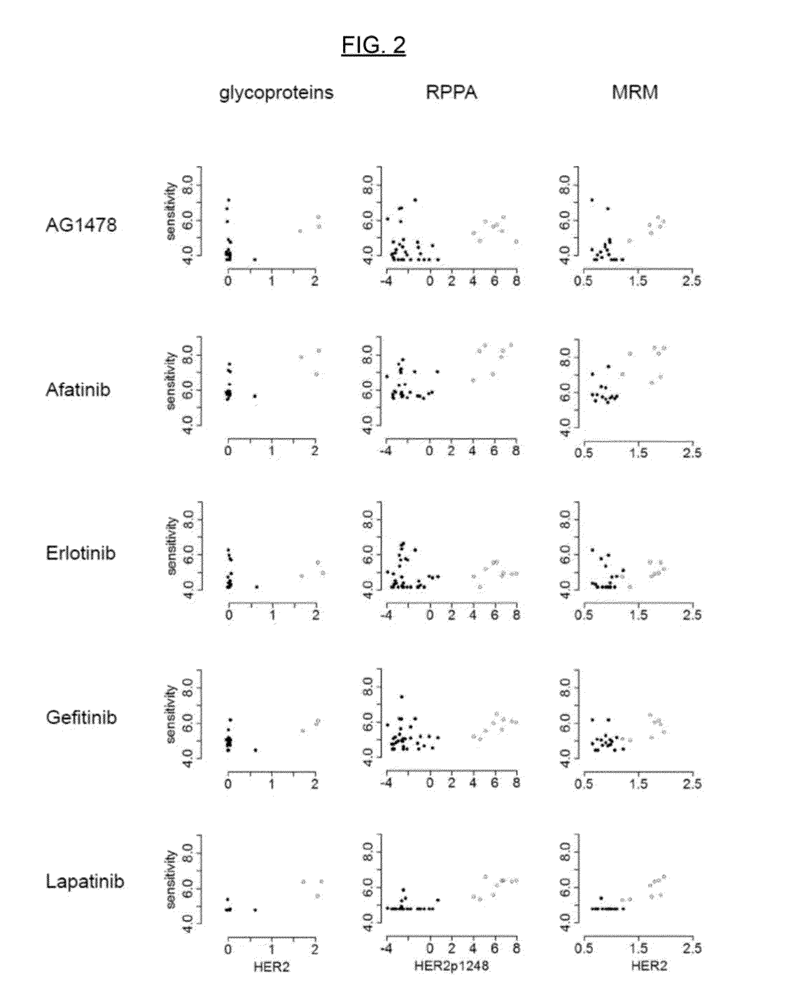

FIG. 2 shows the relation between HER2 expression and the drug sensitivity for the five EGFR or HER2 inhibiting drugs. Sensitivity is the negative base ten logarithm of the 50% growth inhibition concentration. In the RPPA dataset, which has the most cell lines, the HER2 overexpressing lines are clearly separated from the others. This difference was used to define HER2 overexpression (e.g., represented by open circles), and the same definition of overexpression versus normal expression is used in the other protein datasets and in other Figures herein. For the glycoprotein and MRM datasets, the base ten logarithms are given on the horizontal axes. The quantitative relationships between HER2 expression levels and drug sensitivities in FIG. 2 are shown in scatterplots, in which each point corresponds to a particular cell line. The protein datasets all include cell lines that overexpress HER2. These cell lines are generally clustered on the right sides of the plots as separate groups. For lapatinib and afatinib, the HER2 over-expressing cell lines have comparatively high drug sensitivity (see, e.g., the vertical axes of FIG. 2).

All three datasets provide evidence of HER2 overexpression in a subset of cell lines. The lapatinib and afatinib data shows that HER2 overexpressing cell lines are among those most sensitive to repression of cell proliferation by lapatinib and afatinib. For both drugs (lapatinib and afatinib), there are also examples (e.g., another subset) of cell lines that are sensitive to (i.e., the proliferation of which can be stopped or repressed by) lapatinib and afatinib, but that do not overexpress HER2. The cell lines do not bear activating mutations of the EGF receptor or HER2. EGFR over-expression therefore appears to contribute to the sensitivity to erlotinib, but not to the other EGFR inhibitor drugs.

HER2 was measured quantitatively in the three independent datasets (glycoprotein, RPPA and MRM) using mass spectrometry, RPPA and MRM, respectively. Since relatively high HER2 expression was not detected in the cell lines that do not overexpress HER2, these cell lines do not provide false negatives with regard to HER2 expression. If the same were to hold for tumors, there would be patients who are not classified as having a HER2 over-expressing cancer, but who would benefit from lapatinib treatment or therapy, and perhaps from treatment or therapy including pertuzumab and/or trastuzumab.

There are also some cell lines with high drug sensitivity that have normal HER2 expression. For example, the cell lines most sensitive to AG1478, erlotinib and gefitinib have normal, rather than high, HER2 expression. Thus, in such cases, HER2 overexpression does not predict drug sensitivity. Drug-sensitive cell lines with normal HER2 expression produce plots with a lopsided V shape. In addition, FIG. 2 shows that for the drug afatinib, the entire pattern of points is shifted up compared to the other drugs (i.e., the cell lines are most sensitive to afatinib).

EGFR (or EGFRp1068) was detected by lasso regression analysis for each of the five drugs in the RPPA dataset, as shown in FIG. 1. Although EGFR was detected in some cell lines in the glycoprotein dataset, it was not analyzed due to low expression levels as measured by spectral counts. EGFR is not present in the MRM dataset.

For the glycoprotein and MRM datasets, the lasso regression method is highly sensitive, correctly identifying HER2 as a predictor biomarker for afatinib and lapatinib effectiveness. Specificity is also relatively good on the glycoprotein and MRM datasets, with only a few false positives in the glycoprotein data. For the RPPA dataset, lasso regression was highly sensitive but not very specific, as there are some false positives. For example, the quantitative data of FIG. 2 show that HER2 is not a predictor for sensitivity to (or effectiveness of) AG1478, erlotinib or lapatinib, yet it was identified as a predictor for those drugs in the RPPA dataset, as shown in FIG. 1.

As a result, lasso regression readily identified HER2 as a predictor or biomarker in the protein and glycoprotein datasets, and EGFR as a predictor or biomarker in one of the protein datasets (i.e., the RPPA dataset). In addition, the comparison of drug sensitivities and HER2 expression levels gave results consistent with some basic known facts. For example, HER2 predicts an effective response to lapatinib, but not to erlotinib or gefitinib, although breast cancer cell lines are generally expected to be sensitive to afatinib, an irreversible blocker of the EGF receptor. These findings demonstrate the utility of lasso regression on protein or glycoprotein expression data and drug response data, as well as the quantitative interpretation of the protein and glycoprotein expression levels.

Several factors may explain the presence of cell lines with low HER2 expression, but high sensitivity to EGFR or HER2 inhibiting drugs. For example, gefitinib and erlotinib are particularly effective blockers of EGF receptors that contain activating mutations. Another potential explanation is that there is copy number variation in the EGFR gene, leading to differences in drug sensitivity. In the RPPA dataset, there is variation in EGFR expression, although less than that for HER2. For erlotinib, the coefficient of correlation between EGFR and sensitivity is 0.59 (p<10⁻⁴). However, for the other four drugs in FIG. 1, the correlations are not significant.

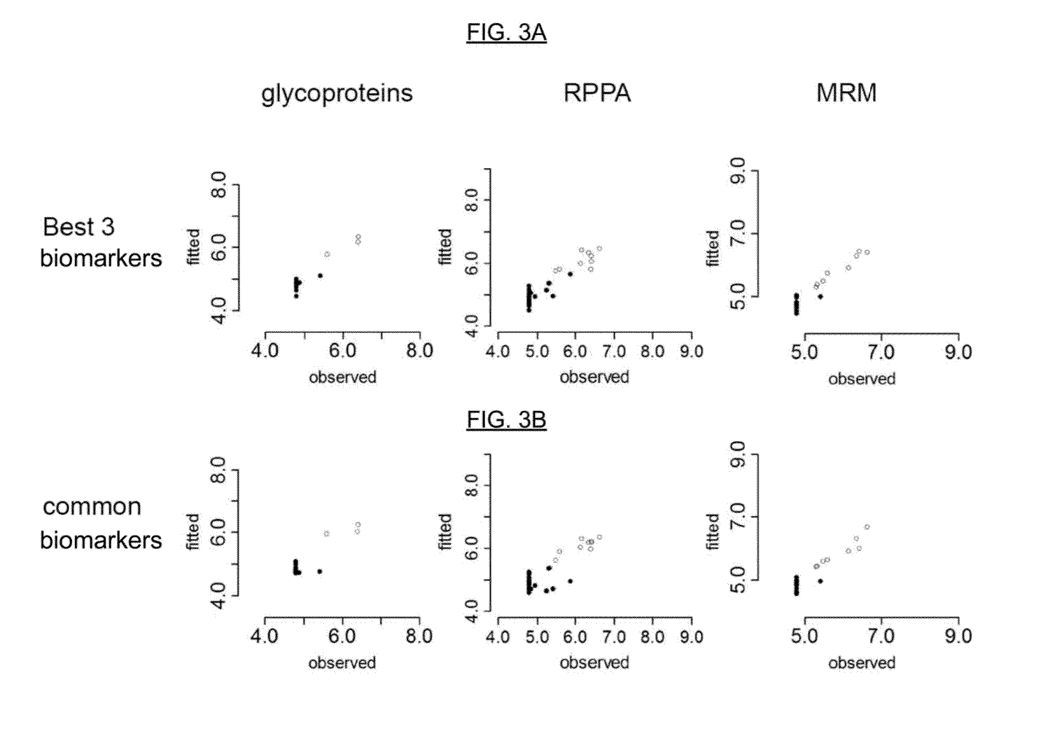

In various embodiments of the present invention, regression systems or models with multiple variables for EGFR blockers may be used to identify drugs effective to stop or repress proliferation of cancer cells. By creating regression models with HER2 (or HER2p1248) and one or two additional predictor variables, it is possible to fit the drug sensitivities to protein and glycoprotein predictors (i.e., biomarkers) using models that are linear in all of the variables. That is, the models can fit the lopsided V shapes of the drug sensitivity relations. For example, fits for lapatinib-sensitive cancer cells using protein or glycoprotein biomarkers are shown in FIGS. 3A-B.

FIGS. 3A-3B show fitted lapatinib sensitive cancer cells using two or three protein predictors. The graphs of FIG. 3A show the best models. The identified biomarkers were (i) HER2, gamma-interferon-inducible lysosomal thiol reductase and neuroplastin from the glycoprotein dataset; (ii) HER2, S6p240 244 and JNKp183 5 from the RPPA dataset; and (iii) HER2, glutathione synthetase and vesicle trafficking protein SEC22b from the MRM dataset, with model R² values of 0.90, 0.87 and 0.92, respectively. The graphs of FIG. 3B show models with biomarkers common to at least four of the five EGFR inhibitor drugs. The glycoprotein dataset was also modeled using HER2, sushi domain-containing protein 2 and BST2 (UniProt accession Nos. PO4626, Q9UGT4, Q10589), with model R²=0.81. The RPPA dataset was also modeled by HER2 and p38, with model R²=0.78. A model using HER2, PNMT and ASCL1, as biomarker indicators, fits the MRM dataset with R²=0.90.

Fitting the drug sensitivity data with two or three biomarkers can provide models or systems that describe sensitivity to EGFR inhibitors of both the HER2 overexpressing and non-overexpressing cell lines. Identifying glycoprotein and/or protein and/or protein biomarkers in assayed tumor samples has the potential to identify additional patients who may benefit from treatment with lapatinib, even if their particular cancer is not one that overexpresses HER2.

The best linear model with three predictors (HER2, gamma-interferon-inducible lysosomal thiol reductase, and neuroplastin) from the glycoprotein dataset had a multiple correlation coefficient R² (e.g., model R²) of 0.90 (see, e.g., the graphs in FIG. 3A). The best three-predictor models from the RPPA and MRM datasets have model R² values of 0.87 and 0.92, respectively. For all three models, the pattern of points in the scatterplot is linear. Adding additional predictor variables allows the cell lines that are highly sensitive to lapatinib, but have normal expression of HER2, to be modeled, as well as the remaining cell lines.

Choosing the best three-predictor models increases the probability of overfitting. Using relatively small numbers of cell lines, it possible that a protein in a dataset complements HER2 by chance. There may be proteins other than HER2 that are over-expressed or under-expressed in the cell lines that are sensitive to the drugs that target EGFR. To reduce overfitting, predictors other than HER2 can be identified independently for these five EGFR inhibitor drugs, as well as possibly others. There are several common biomarker predictors in addition to HER2. For example, sushi domain-containing protein 2 (SUSD2) is a biomarker for all five drugs.

As shown in FIG. 3B, a model with HER2, SUSD2, and bone marrow stromal protein 2 (BST2) as biomarkers for HER2 or EGFR inhibitor drugs using the glycoprotein database fits the lapatinib sensitivities with a model R² of 0.81. In the RPPA dataset, p38 and cleaved caspase 7, in addition to HER2p1248, were predictors for all five HER2 or EGFR inhibitor drugs. A model with HER2 and p38 fits the lapatinib sensitivities with model R² of 0.78 (see, e.g., the center graph in FIG. 3B). However, adding cleaved caspase 7 does not provide any appreciable improvement. In the MRM dataset, phenylethanolamine N-methyltransferase (PNMT) and long chain fatty acid CoA ligase 1 (ACSL1) both are predictors for all five HER2 or EGFR inhibitor drugs. HER2, PNMT, and ACSL1 provide a model with R² of 0.90 (see, e.g., the right-hand graph in FIG. 3B).

The glycoprotein and protein datasets have few proteins in common, so it is expected that the variables added to HER2 will be different in each different dataset. Among the datasets analyzed, a “common biomarkers” model does not provide the best results for a three-biomarker effectiveness predictor set in the statistical analysis. However, a three-biomarker model in practice (e.g., diagnosis or treatment of cancer using one of the three-biomarker sets identified in one or more protein or glycoprotein databases) has the advantage that biomarkers can be found for most EGFR inhibitor drugs, increasing the likelihood that they have biological significance. These results show that a small number of biomarkers (e.g., 1, 2, or 3) can provide a relatively good correlation between the fitted and observed drug sensitivities, regardless of whether dataset-specific biomarkers or biomarkers common to multiple or all analyzed datasets are used.

Exemplary Specific Biomarkers for EGFR and HER2-Targeting Drugs

Several drugs target epidermal growth factor receptor (EGFR; the official gene name is ERBB1) and human epidermal growth factor receptor 2 (HER2; the official gene name is ERBB2). For example, afatinib is used in treating lung cancer and is in clinical trials for treating breast cancer. Erlotinib (e.g., Tarceva) is currently used in treating lung cancer. Gefitinib (e.g., Iressa) is currently used in treating lung cancer). Lapatinib (e.g., Tykerb) is used in treating breast and lung cancer. Using one or more of the three example protein or glycoprotein datasets described herein, specific biomarkers associated with or correlated to effectiveness of a drug to stop or repress proliferation or growth of one or more types of cancer cells were identified by lasso regression analysis.

For afatinib, the glycoprotein biomarker(s) may include receptor tyrosine-protein kinase erbB-2 (PO4626), cathepsin B (P07858), cadherin-13 (P55290), bone marrow stromal antigen 2 (Q10589), and/or sushi domain-containing protein 2 (Q9UGT4). For erlotinib, the glycoprotein biomarker(s) may include sushi domain-containing protein 2 (Q9UGT4), neprilysin (P08473), large neutral amino acids transporter small subunit 1 (Q01650), integrin alpha-6 (P23229), dipeptidyl peptidase 1 (P53634), collagen alpha-1 (VI) chain (P12109), and/or neutral amino acid transporter B (Q15758). For gefitinib, the glycoprotein biomarker(s) may include transcobalamin-1 (P20061), sushi domain-containing protein 2 (Q9UGT4), podocalyxin (000592), large neutral amino acids transporter small subunit 1 (Q01650), laminin subunit beta-1 (P07942), and/or dipeptidyl peptidase 1 (P53634). For lapatinib, the glycoprotein biomarker(s) may include receptor tyrosine-protein kinase erbB-2 (PO4626), gamma-interferon-inducible lysosomal thiol reductase (P13284), neuroplalstin (Q9Y639), cathepsin B (P07858), CD44 antigen (P16070), and/or bone marrow stromal antigen 2 (Q10589).

Predictive Biomarkers for AKT1,2 Inhibitors

FIG. 4 shows fitting of AKT1 inhibitors with AKTp478 and PDK1, as biomarkers. The RPPA proteins include AKT (AKT1), AKTp473 and PDK1 (a kinase that phosphorylates AKT). AKTp473 and PDK1 (or PDK1p241) were identified as biomarkers by lasso regression for all three drugs. The Sigma AKT1,2 inhibitor is modeled using only PDK1 as a biomarker, and the others (GSK2141795 and triciribine) with PDK1 and AKTp478. A regression model with AKTp473 and PDK1 as biomarkers allowed the fitting of the GSK2141795 sensitivities with multiple correlation coefficient R²=0.52 (see FIG. 4). By itself, PDK1 gives a better single biomarker model (R²=0.36) than does AKTp473 (R²=0.20). For the Sigma AKT1, 2 inhibitor, PDK1 as a single biomarker gives a model with R²=0.48. Adding AKTp473 does not improve the model. The range of observed drug sensitivities was significantly lower for the Sigma AKT1,2 inhibitor than for GSK2141795. Modeling triciribine sensitivity failed with AKTp478 and PDK1. For the AKT1 inhibitor drugs lasso regression successfully found both the nominal drug target and a modulator, PDK1. For the AKT1 inhibitor drugs, in which modeling succeeded, PDK1 is a more useful biomarker, even though AKT is the nominal target.

While AKT was found as a candidate biomarker for two AKT inhibitors (GSK2141795 and Sigma AKT1,2 inhibitor), the AKT phosphorylating enzyme PDK1 was more useful as a single biomarker in regression models. Thus, AKT may be useful as a biomarker in three-biomarker models for drugs targeting an AKT1,2 inhibitor.

One- and Three-Biomarker Predictive Models

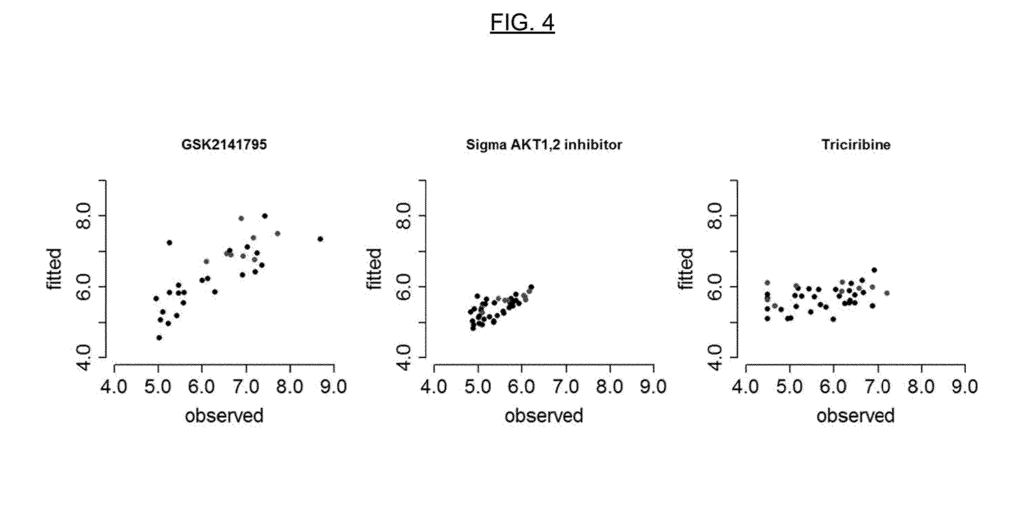

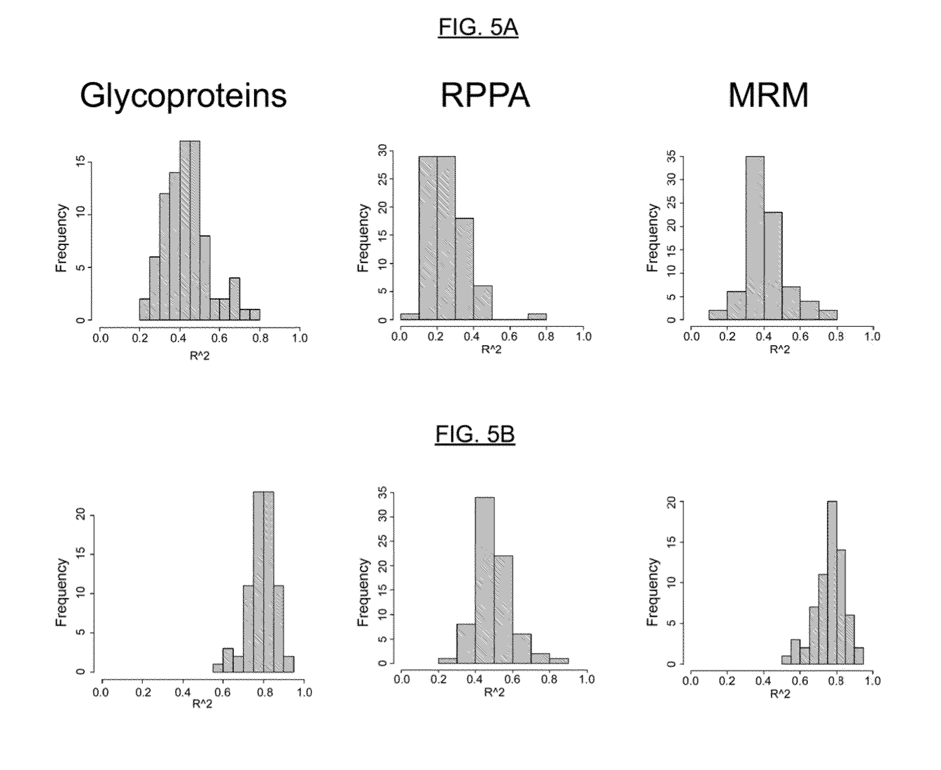

FIGS. 5A-5B show frequency distributions of multiple coefficient of correlations R² for single biomarker models (FIG. 5A) and three-biomarker models (FIG. 5B). The model R² values between the observed and fitted drug sensitivities varied from 0 to nearly 0.8 in the single biomarker models (FIG. 5A). The frequency distributions of the model R² values for the glycoprotein, RPPA and MRM datasets are all unimodal and approximately symmetrical, as expected from statistical theory. The significance of the models was evaluated with an overall F test, in which the null hypothesis is that the regression coefficient for the single biomarker is zero. In the glycoprotein and MRM datasets, all p values were less than 0.05, and the majority of the p values were less than 0.01. In the RPPA dataset, the model for one drug, FTase inhibitor 1, had a p value higher than the conventional 0.05 level of significance, and the other models had p values lower than the conventional 0.05 level of significance. Each of the distributions in the single biomarker models is skewed slightly to the right due to a few drugs for which an especially good model was found (FIG. 5A).

Twelve drugs with corresponding single biomarkers from each of the three datasets are shown in FIG. 6. These twelve single biomarkers models for the three datasets were determined according to the statistical analysis method(s) described herein. In all three datasets, HER2 (or HER2p1248) and lapatinib are the best single biomarker/drug pair. In the glycoprotein dataset, large neutral amino acids transporter small subunit 1 (SLC7A5) is a useful single biomarker for erlotinib, gefitinib and AG1478. The best single biomarker for GSK2141795 and the Sigma AKT inhibitor is PDK1, as discussed above. In the MRM dataset, anterior gradient protein 2 homolog (AGR2) is a biomarker for the same two AKT inhibitors (e.g., AKTp478 and PDK1). Finally, in the RPPA dataset, IGFBP2 is a useful single biomarker for paclitaxel and docetaxel, which are similar chemically and functionally.

For each drug, the best one-biomarker and three-biomarker linear models were found using the Leaps and Bounds algorithm (Furnival, G M and Wilson, R W J, “Regressions by leaps and bounds,” Technometrics, 2000; 42:69-79). The best single biomarker was usually one of the protein or glycoprotein predictors identified with high probability. The R² values for the models with three biomarker variables are higher in general than they are for the one-biomarker models (compare FIG. 5B with FIG. 5A). For the glycoprotein and MRM datasets, the average single biomarker model R² values were 0.44 and 0.41, respectively. Models constructed from the RPPA biomarkers did not work quite as well, with an average R²=0.26. In the best three-biomarkers models, the average R² values were 0.79, 0.50, and 0.76 for biomarkers identified in the glycoprotein, RPPA and MRM datasets, respectively. Under the overall F test, all three-biomarker models had p values <0.01. Increasing the number of biomarkers may improve performance in fitting the observed drug sensitivities and in distinguishing the corresponding effective drug from other drugs that may be associated with one or more common biomarkers. The magnitude of the improvement is greater for the measurements made with mass spectrometry than with RPPA. Measurements made with RPPA rely on the amount, number or density of antibodies and on densitometry for quantification of protein levels, which makes quantified results determined from RPPA analysis less reliable than results determined from mass spectrometry.

Comparison of Glycoprotein Expression Levels with mRNA Expression Levels as Biomarkers

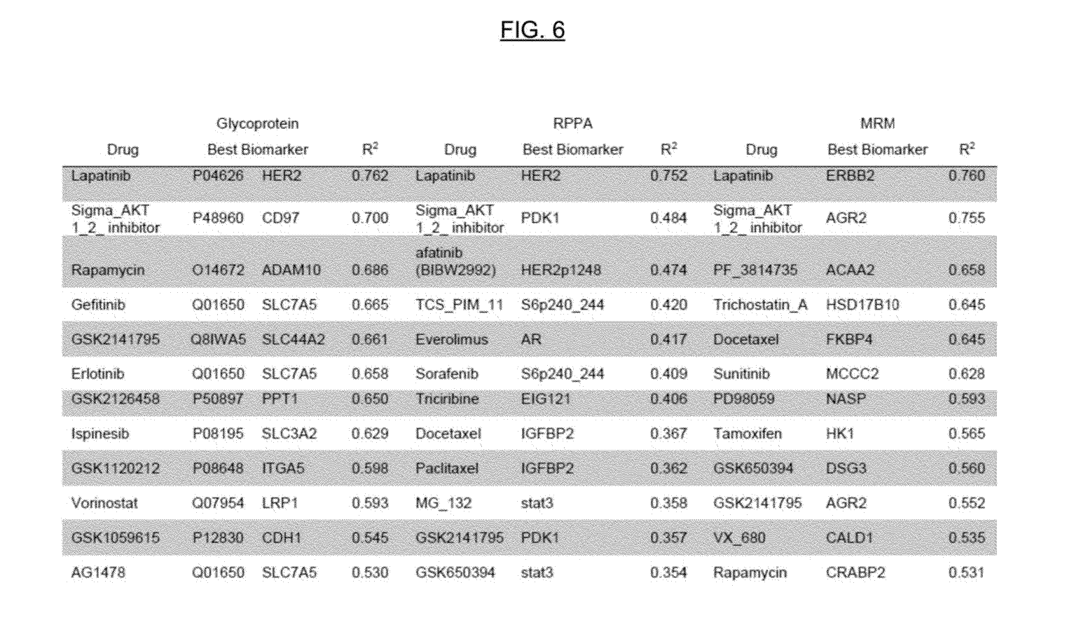

FIG. 7 shows an association between glycoprotein expression levels and the corresponding mRNA levels, measured for 185 glycoprotein/mRNA in pairs in 19 cell lines. The base₂ logarithms are plotted. Using mRNA expression levels as predictor variables generally does not provide results similar to those of glycoprotein expression levels as biomarkers.

RNA sequence data is available for many of the same breast cancer cell lines analyzed in the glycoprotein dataset. From that data, one can find the RNA expression levels for 185 glycoproteins in 19 cell lines included in the glycoprotein dataset. Lasso regression was carried out as described herein on this mRNA data.

There were a total of 1473 biomarkers identified for all drugs in the mRNA data, compared to 1430 for the glycoprotein data. In 237 cases, the mRNA and corresponding glycoprotein were found as biomarkers for the same drug. For a few drugs, such as lapatinib and trametinib (GSK1120212), the best predicting mRNA and protein are the same. Trametinib (GSK1 120212) has recently been approved for use in metastatic melanoma with certain BRAF mutations. However, the overall correlation between the glycoprotein and RNA sequence data, if any, is relatively modest. One reason is that the mRNA and glycoprotein expression levels have a relatively weak correlation with each other, as shown in FIG. 7. As a result, mRNA expression levels do not predict expression levels very well in the glycoprotein dataset.

Cross-Validation of the Identified Biomarkers

Leave-one-out cross-validation was used to address whether systems or models generated with lasso regression may be expected to work on cell lines assayed in various labs for the glycoprotein dataset (e.g., prediction error). For many drugs, the number of cell lines n was 22. In cases with incomplete drug data, n was lower. For each drug, the cross-validation was performed n times (i.e., each cell line was left out once). The best one, two, or three predictor models were rebuilt on the same predictor proteins using the n−1 cell lines and ordinary least squares regression. The drug sensitivity of the left-out cell line was predicted using the rebuilt model. The cross-validation error was measured by the mean of the n squared prediction errors (MSPE).

Cross-validation shows that there can be variation in the prediction error for different drugs. One factor influencing the prediction error is the range of sensitivities of the cell lines to each drug, which varies considerably. For those drugs with relatively small prediction errors after suitable normalization, lasso regression may perform relatively well on data from cell lines not used to create the model.

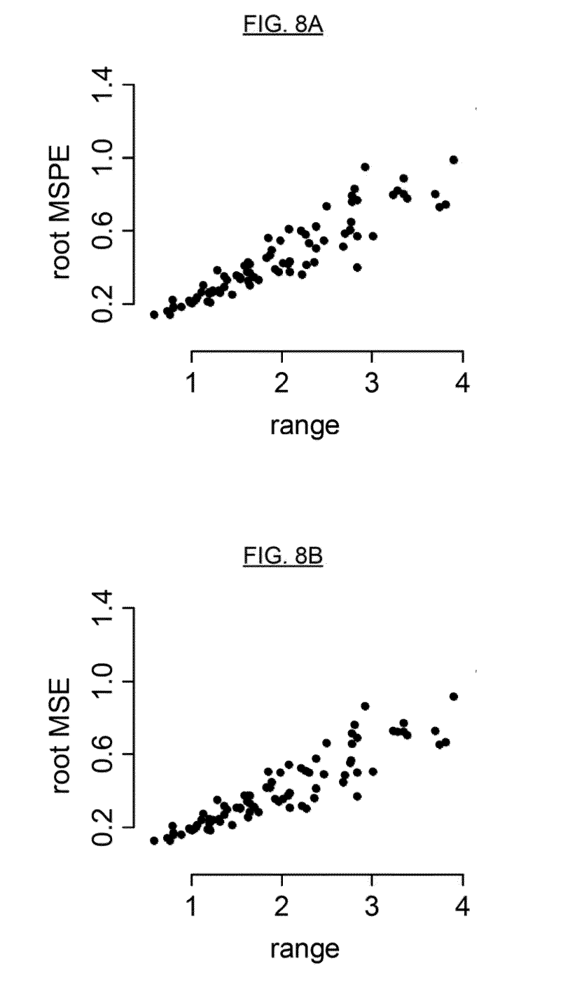

FIG. 8A shows the root MSPE and FIG. 8B shows the root MSE for the leave-one-out cross-validation experiment, which are both proportional to the range of drug sensitivities. The range for a drug is the difference in sensitivities between the most sensitive and least sensitive cell lines in the group of analyzed cell lines. There is a strong association between the root of the MSPE (the mean of the n squared prediction errors), which was calculated with one cell line left, and the range of sensitivities for a given drug (see, e.g., FIG. 8A). The mean square error (MSE) calculated with no cell line left out for a drug was also correlated with the range of sensitivities (see, e.g., FIG. 8B). To control for drug sensitivity range as a factor in prediction error, the MSPE/MSE ratios for each of the 90 drugs analyzed were found. As a result, prediction error for each of the 90 drugs analyzed has been accurately accounted for.

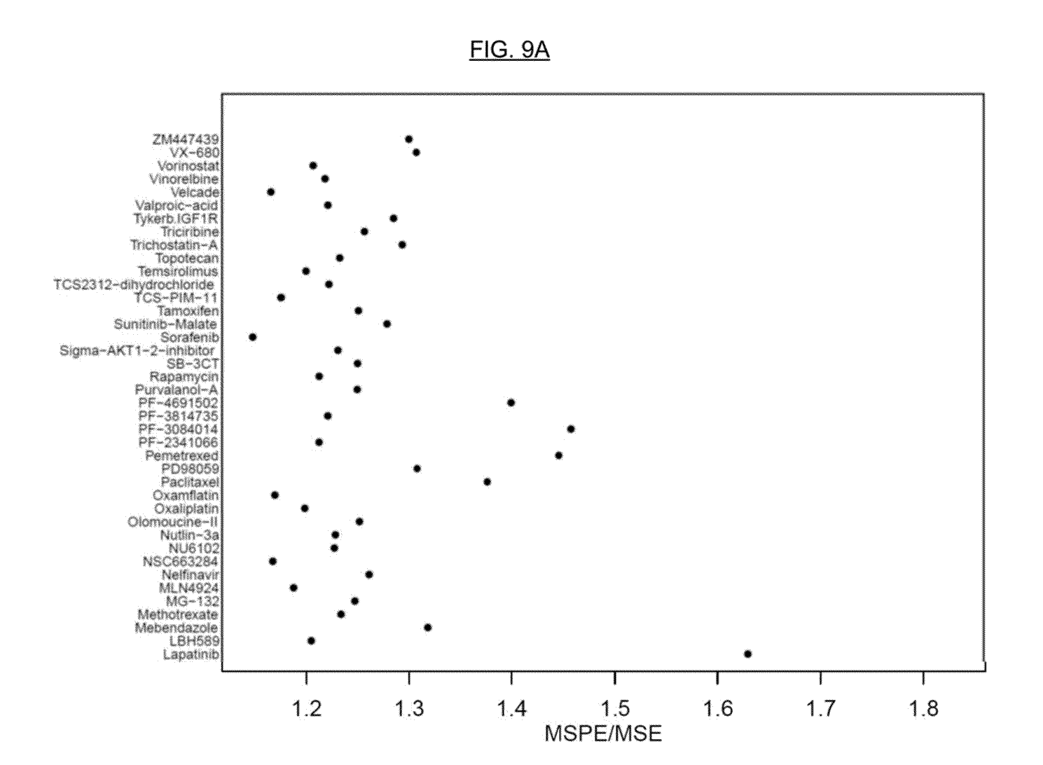

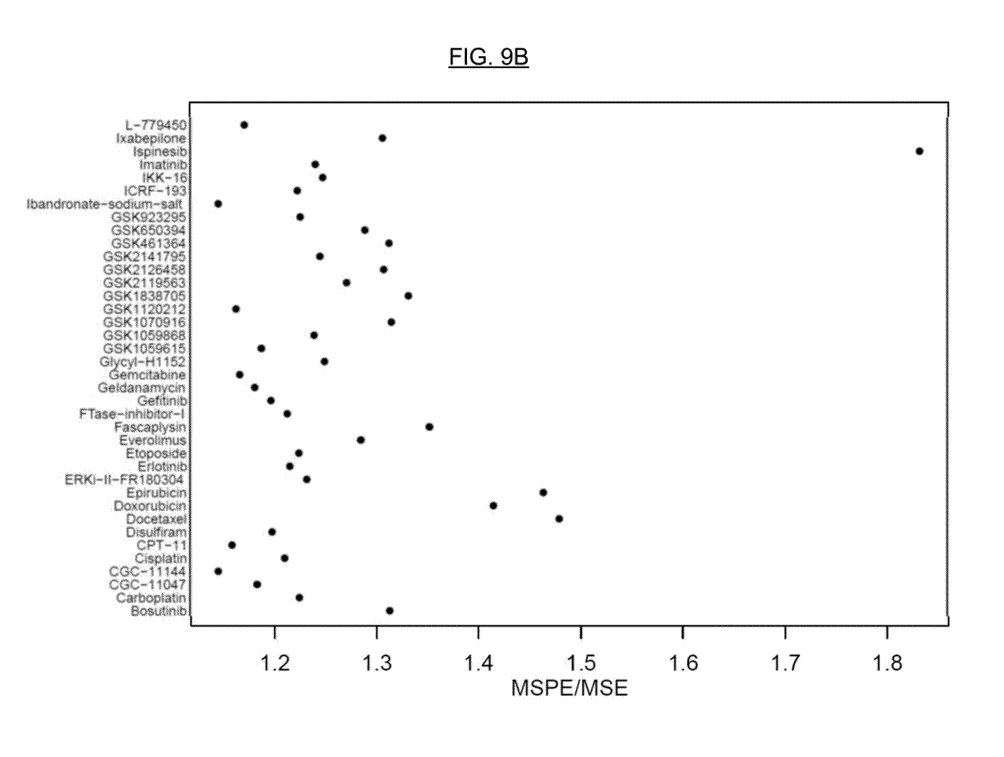

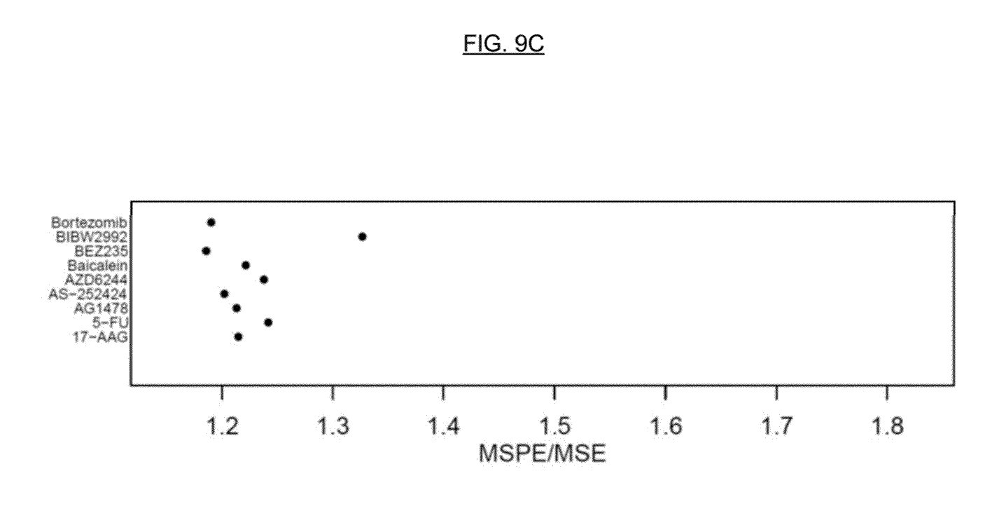

FIGS. 9A-C show estimates of prediction error relative to mean square error for the best models with one biomarker. If the prediction errors were the same size as the errors in the training set, the MSPE/MSE ratio would be 1. Most of the observed ratios fall between 1.1 and 1.3. For a given drug, as the number of biomarkers increases to three, the MSPE and MSE values generally decline as the fits improve.

The two drugs with the highest MSPE/MSE ratios are ispinesib and lapatinib. In both cases, the drug has a few outliers. For example, with lapatinib, the three HER2 overexpressing cell lines with high HER2 are outliers. HER2 was not detected in the other cell lines. When the HER2 overexpressing cell lines are left out in the cross-validation, the effect on the regression coefficients for the training set is large, and leads to large prediction errors. This sort of error would be expected to decline in a larger dataset, especially one with more HER2 over-expressing cell lines.

Predictive Biomarkers for mTOR Inhibitors

mTOR inhibitors, such as rapamycin, everolimus and temsirolimus, are related compounds that block the mammalian target of rapamycin (mTOR). Rapamycin (sirolimus) and temsirolimus are anti-proliferative drugs with similar mechanisms of action. Temsirolimus is in multiple clinical trials for advanced solid tumors. Everolimus is approved for use in patients with ER+, HER2− breast cancer, in combination with exemestane. The cell lines analyzed varied in sensitivity to these drugs over 4.6, 3.3, and 3.7 orders of magnitude, respectively.

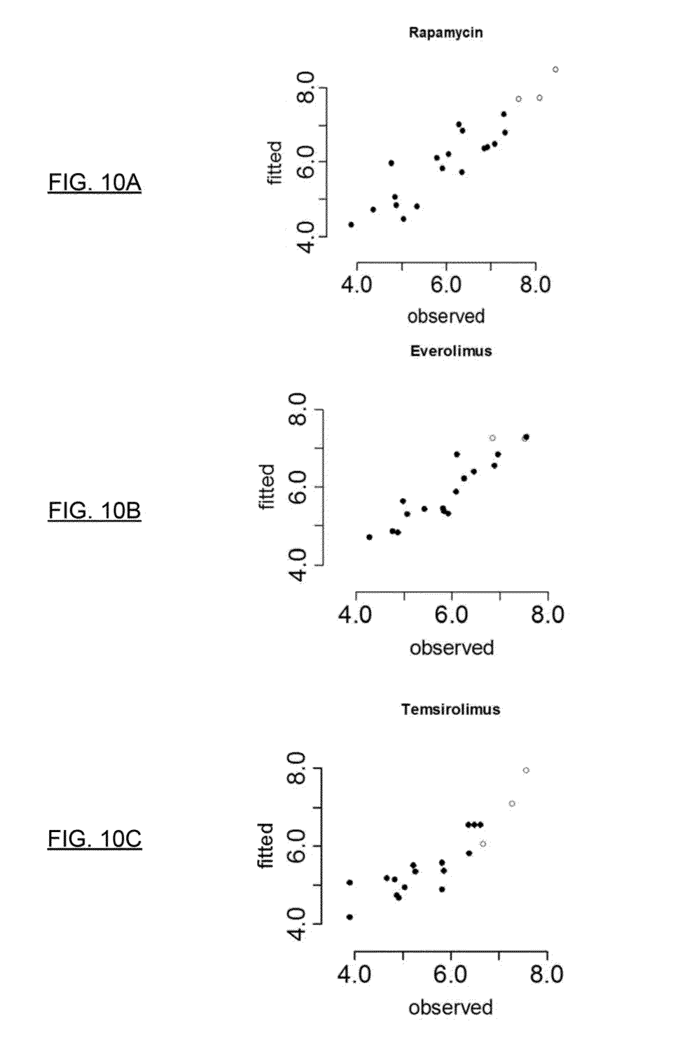

FIGS. 10A-C show modeling of the mTOR inhibitors, rapamycin, everolimus and temsirolimus. The biomarkers associated or correlated with rapamycin may include disintegrin and metalloproteinase domain-containing protein 10 (014672), V-set domain-containing T-cell activation inhibitor 1 (Q7Z7D3), and/or pituitary tumor-transforming gene 1 protein-interacting protein (P53801). The biomarkers associated or correlated with everolimus may include insulin-like growth factor-binding protein 7 (Q16270), lysosomal pro-x carboxypeptidase (P42785) and/or receptor tyrosine-protein kinase erbB-2 (PO4626). The biomarkers associated or correlated with temsirolimus may include transmembrane emp24 domain-containing protein 7 (Q9Y3B3), arylsulfatase A (P15289) and/or receptor tyrosine-protein kinase erbB-2 (PO4626).

mTOR is in the RPPA dataset, but was identified with very low probability as a biomarker for the mTOR inhibitor drugs analyzed. All three mTOR inhibitors analyzed are modeled relatively well with three glycoprotein biomarkers, as shown in FIGS. 10A-C. HER2 over-expressers are among the most sensitive cell lines. As a result, HER2 (i.e., erbB-2 and/or PO4626) is a biomarker for everolimus and temsirolimus.

Predictive Biomarkers for Taxanes and Standard Chemo-Resistant Cancers

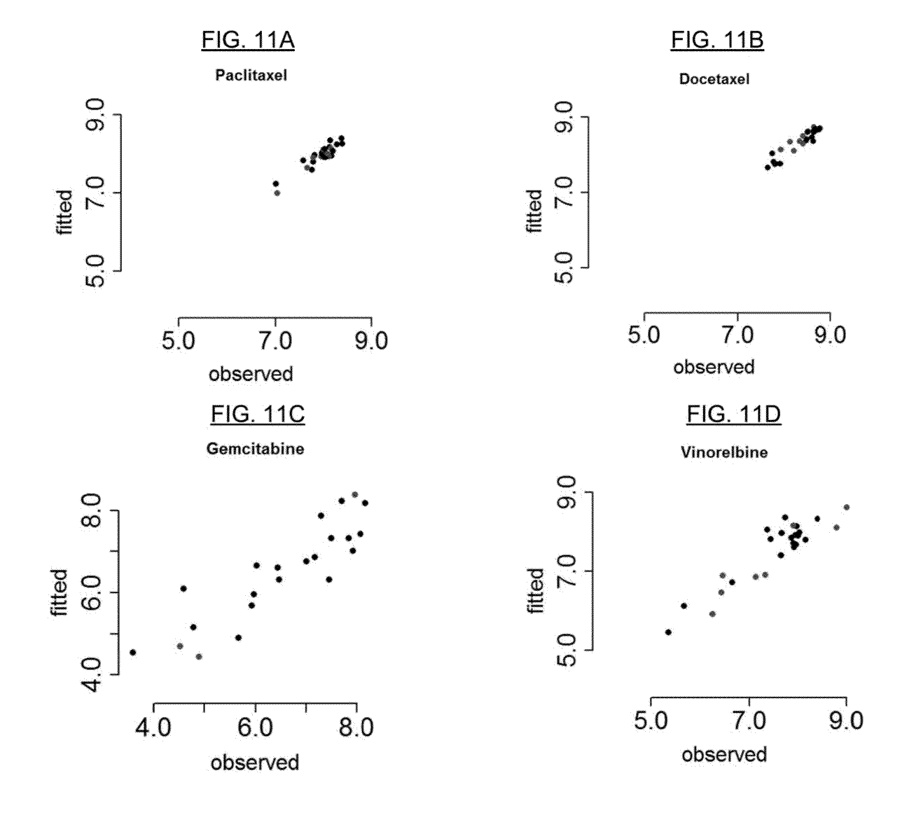

Drugs that target microtubules include taxanes (e.g., paclitaxel and docetaxel). Gemcitabine and vinorelbine are drugs used for patients who experience recurrence of cancer after treatment with the standard of care chemotherapy and/or with taxanes. Gemcitabine is a nucleoside analogue that targets nucleic acids (e.g., DNA). A molecular target of vinorelbine is also tubulin. Using the methodology disclosed herein, FIG. 11 shows modeling of taxanes (e.g., paclitaxel and docetaxel), gemcitabine and vinorelbine, according to exemplary embodiments of the present invention.

The protein biomarkers associated or correleated with paclitaxel includes one or more of ubiquitin carboxyl-terminal hydrolase 5 (USPS; P45974), solute carrier family 2, facilitated glucose transporter membrane 1 (SLC2A1; P11166), and alpha-aminoadipic semialdehyde dehydrogenase (ALDH7A1; P49419) from the MRM dataset as candidate biomarkers. Alternatively or additionally, the biomarker may include one or more of the following glycoproteins: CD276 antigen (Q5ZPR3), cathepsin Z (Q9UBR2), and serpin H1 (P50454). The biomarkers for docetaxel may include one or more of the following proteins: lysosome membrane protein 2 (SCARB2; Q14108), alpha-aminoadipic semialdehyde dehydrogenase (ALDH7A1; P49419) and isochorismatase domain-containing protein 1 (ISOC1; Q96CN7) from the MRM dataset. Alternatively or additionally, the biomarker may include at least one of the following glycoprotein(s): beta-mannosidase (000462), cathepsin Z (Q9UBR2), and serpin H1 (P50454).

The response to paclitaxel varies widely in breast cancer patients. Thus, predictive biomarkers for response to paclitaxel may be valuable. Docetaxel, a derivative of paclitaxel, is used as a component of combination chemotherapy in treatment of breast cancer. The sensitivity of the cell lines to paclitaxel and docetaxel varied over a much smaller range than the sensitivity of the cell lines to rapamycin. For both paclitaxel and docetaxel, predictive models with high model R² were discovered. The best predictors for paclitaxel were found in the MRM dataset, such as ubiquitin specific peptidase 5 (USPS), facilitative glucose transporter (SLC2A1), and aldehyde dehydrogenase (ALDH7A1).

The biomarker(s) associated or correlated with vinorelbine may include one or more of the following proteins identified in the MRM dataset: glucose-6-phosphate 1-dehydrogenase (P11413), ribonuclease UK114 (P52758), and tropomyosin alpha-4 chain (P67936). The glycoprotein biomarker(s) associated or correlated with gemcitabine and possible other drugs that target one or more nucleic acid (e.g., DNA) may include ganglioside GM2 activator (P17900), granulins (P28799), and/or steryl-sulfatase (P08842). For gemcitabine and vinorelbine, the biomarkers may alternatively or additionally include one or more of G6PD (P11413), HRSP12 (P52758) and TPM4 (P67936) from the MRM dataset, respectively.

The best model for gemcitabine (R²=0.77) was identified in the glycoprotein dataset, with biomarkers including ganglioside GM2 activator (P17900), granulins (P28799) and steryl sulfatase (P08842). The best model for vinorelbine (R²=0.85) was identified in the MRM dataset, with biomarkers including glucose-6-phosphate 1-dehydrogenase (G6PD), ribonuclease UK114 (HRSP12), and tropomyosin alpha-4 chain (TPM4).

As with rapamycin, the sensitivities of the cell lines to gemcitabine and vinorelbine spanned approximately four orders of magnitude. If this variation reflects the situation in patients' tumors, there are patients who are highly sensitive to these drugs, and more predictive or effective biomarkers may be useful to identify those cancers that are more likely to be treated effectively with gemcitabine and vinorelbine.

Predictive Biomarkers for PI3′ Kinase Inhibitors

PI3′ kinase inhibitors, such as AS-252424, BEZ235, GSK1059615, GSK2119563, GSK2126458 and PF 4691502 target PI3′ kinase. In some cases, these PI3′ kinase inhibitors also target rapamycin (mTOR). Another drug that targets or inhibits PI3′ kinase is BEZ235, which is in clinical trials for breast cancer. Using the methodology disclosed herein for identifying one or more biomarkers associated or correlated with effective repression of cancer cells, the glycoprotein biomarker(s) identified may include one or more of collagen alpha-1 (VI) chain (P12109), large neutral amino acids transporter small subunit 1 (Q01650), mucin-1 (P15941), and/or receptor tyrosine-protein kinase erbB-2 (PO4626).

The catalytic subunit of PI3′ kinase, p110, is in the RPPA dataset, but was not identified as a predictor for these drugs. However, PTEN, which catalyzes the reverse reaction, was identified as a predictor with high probability for 4 out of the 6 non-BEZ235 PI3′ kinase inhibitor drugs (i.e., AS-252424, BEZ235, GSK1059615, GSK2119563, GSK2126458 and PF 4691502). Thus, PTEN may be a useful single protein biomarker for PI3′ kinase inhibitors. For all of these drugs, HER2 overexpression is associated with sensitivity.

Predictive Biomarkers for CDK Inhibitors

CDK inhibitors, such as fascaplysin, NU6102, Oloumucine II, Purvalanol, and palbociclib, are inhibitors of cyclin-dependent kinases (CDKs). Palbociclib is in clinical trials for breast cancer. For all of these drugs except Oloumucine II, one or more cyclins were identified in the RPPA dataset as a biomarker in a high proportion of lasso runs. The best model for palbociclib, with R²=0.79, was identified in the MRM protein dataset, and identified mitochondrial thioreduxin-dependent peroxidide reductase (PRDX3; P30048), acyl-amino acid releasing enzyme (APEH or ApeH-1; Q97YB2), and importin subunit alpha (KPNA2; P52292) as protein biomarkers. However, other protein biomarker(s) associated or correlated with palbociclib may include G2/mitotic-specific cyclin-B1 (P14635; CCNB1), G1/S-specific cyclin-E1 (P24864; CCNE1), identified in the RPPA dataset.

For both PI3′ kinase inhibitors and CDK inhibitors, the nominal target may not be known with absolute certainty, but biomarkers can be identified for such drugs. For these drugs, proteins functionally related to PI3′ kinase and CDK, such as phosphatase and tensin homolog (PTEN) and cyclins, may be useful as biomarkers.

Predictive Biomarkers for Drugs that Target Lung Cancers

Pemetrexed is approved for use on some lung cancers. A regression analysis was performed on the example glycoprotein dataset for sensitivity to pemetrexed using the exemplary approach disclosed herein. Its effect on proliferation in breast cancer and/or other cancers may be based on a predictive model including the glycoprotein biomarkers liver carboxylesterase 1 (P23141), tetraspanin 1 (060635) and seizure 6-like protein 2 (Q6UXD5).

Summary of Example Drug/Biomarker Associations and/or Correlations

The present lasso regression analysis, which may be related mathematically to ridge and elastic net regression, was used for variable selection, followed by identification of the best model with up to three biomarkers using the Leaps and Bounds algorithm. The approach of the present invention advantageously identifies specific proteins and/or glycoproteins that may be useful as predictive variables (i.e., biomarkers) in regression analysis. Many proteins and glycoproteins can be evaluated quantitatively or semi-quantitatively using standard techniques, such as immunohistochemistry or immunofluorescence.

For 86 of the 90 drugs analyzed, a regression model with at least one glycoprotein predictor variable (ie., biomarkers) and an intercept is significantly better than a model with intercept and no predictor variable. With one predictor variable, models or systems may be generated with high multiple correlation coefficient for several drugs, including lapatinib and the Sigma AKT1,2 inhibitor, as shown in FIG. 11. Adding one or two additional glycoprotein predictor variables (i.e., biomarkers) generates statistically significant models for 87 of the 90 drugs.

The dataset including glycoprotein expression data obtained using mass spectrometry outperformed the RPPA dataset in specificity, and in providing relatively good fits to data. There are several possible reasons for the better performance of the glycoprotein dataset (and the MRM dataset), as compared to the RPPA dataset. First, the RPPA dataset covers more cell lines (47 cell lines) than the glycoprotein dataset (22 cell lines) or MRM dataset (27 cell lines). As a result, three biomarker predictive models are relatively closer to a saturating model for the glycoprotein and MRM datasets, thus providing better results. Second, measurements from mass spectrometry are less noisy than RPPA measurements. Third, the proteins in the RPPA dataset may not vary as much in their expression levels as the proteins and glycoproteins in the other two datasets. The functions of many of the RPPA proteins are known to depend on their state of phosphorylation or their subcellular localization. Perhaps the proteins in the RPPA dataset simply vary less in their expression levels, and thus are less useful for modelling based on quantified expression. A combination of the above mentioned factors may also account for the difference in performance.

The models and/or systems presented above identify many biomarker proteins for predicting drug response in breast cancer cell lines. Increasing the number of predictors from one to three generally leads to an improvement in the reliability of the models or systems, as indicated by model R². Among the analyzed drugs for which there are existing predictive and/or diagnostic models, several drugs are already approved by the FDA for use in breast cancer, whereas others are still being evaluated. Over the approximately 90 analyzed drugs and three different protein and glycoprotein databases, the strongest correlation between a drug and a single biomarker was lapatinib and HER2, with model R² of approximately 0.76 in each of the three datasets. Taking this value of model R² as an estimate or threshold of the quality of fit for a model or system to have clinical utility, when employing two or three biomarkers, the threshold or estimate is met by 57 drugs using the glycoprotein dataset, 1 drug using the RPPA dataset, and 37 drugs using the MRM datasets. Thus, it may be possible to predict patient responses to dozens of drugs for which there are currently no biomarkers by developing new biomarkers based on quantitative measurement of two or three protein or glycoprotein biomarkers.

Exemplary Method(s) of Preparing Samples for Qualitative and Quantitative Expression Level Determination

The protein or glycoprotein biomarkers may be identified or measured in tumor samples by immunohistochemistry, but protein or glycoprotein expression levels may still need to be quantified (e.g., by mass spectrometry) for a more reliable analysis. An advantage of immunohistochemistry is the opportunity to look for potentially confounding changes of expression of a biomarker protein in non-carcinoma cells. A second approach to measuring biomarker proteins and/or glycoproteins in tumor samples may include using targeted assays and mass spectrometry on tumor samples. The MRM dataset is an example of an approach that targets specific proteins. Proteins and glycoproteins may be extracted from formalin-fixed paraffin embedded samples for quantitative analysis by mass spectrometry. For creating models, these samples are readily available, relative to fresh or frozen tissue. Whether immunohistochemistry or mass spectrometry is employed, it is possible to generate predictive biomarkers for many more drugs used in breast cancer and other cancers.

For a glycoprotein dataset, a protocol for glycoprotein enrichment may be used. In one example, mass spectrometry was carried out on a Thermo LTQ ion trap mass spectrometer, and also on a Thermo Q Exactive Orbitrap mass spectrometer. Spectral counts were used for relative expression levels of a given glycoprotein in the different cell lines. The results on aliquots from the same glycoprotein sample were similar in the two spectrometers, although less protein was required for the Orbitrap instrument. To combine samples from the two datasets (the LTQ- and the Orbitrap-generated data), the data were plotted in a quantile-quantile plot, and a line was plotted. Using the slope and intercept, an inverse transform was applied to the data from the Orbitrap Mass Spectrometer, forcing it to have the same center and dispersion as the data from the LTQ Mass Spectrometer.

For HCC1395, HCC1428, HCC38, HMEC3 and MDAMB468, there was data from only the Q Exactive data. For these cell lines, the spectral counts were normalized to the data from the LTQ Mass Spectrometer, using proteins that have the least variation in expression across the various cell lines. The seven glycoproteins in the LTQ Mass Spectrometer dataset with the lowest coefficient of variation were P20645, Q9BT09, P62937, Q16563, Q9BVK6, Q08722, and P07602. The Euclidean length of the spectral counts for these glycoproteins was calculated for both the LTQ Spectrometer data and the Q Exactive Mass Spectrometer data. The ratio was used to normalize all Q Exactive spectral count data. After combining LTQ and Q Exactive data, glycoproteins with fewer than 100 spectral counts over all the cell lines were dropped from the dataset. Base ten logarithms (as in the drug sensitivity data) of the spectral counts were taken for use in regression.

For the RPPA dataset, the data was used as published (Daemen A, Griffith O L, Heiser L M, Wang N J, Enache O M, Sanborn Z, “Modeling precision treatment of breast cancer,” Genome Biol. 2013; 14:R110). For the MRM data, the mass spectrometry data and measurements were collected from three sites, and three replicates were taken at each site (Kennedy J J, Abbatiello S E, Kim K, Yan P, Whiteaker J R, Lin C, “Demonstrating the feasibility of large-scale development of standardized assays to quantify human proteins,” Nat Methods, 2014; 11:149-55). Two peptides were measured per protein. In some cases, the measured values fell outside limits of quantitation. For use in the present invention, the replicates and the data from the different sites were averaged. For each protein, the peptide with the highest signal was selected. In cases in which numerical values were not provided, an appropriate upper or lower limit of quantitation was used. The final dataset includes 325 proteins. Base ten logarithms were used for regression.

Exemplary Methods of Diagnosing and Treating Cancer

The present invention concerns a method of diagnosing and/or treating cancer. The method generally comprises identifying and quantifying at least one biomarker (e.g., one or more protein or glycoprotein biomarkers) in cancer cells from a patient, identifying one or more of a plurality of drugs that effectively stop or repress proliferation of the cancer cells from a correlation or association of the biomarker(s) with effectiveness of the drug(s) to stop or repress the proliferation of the cancer cells, and optionally (e.g., in the method of treating cancer) administering the correlated or associated drug(s) in a pharmaceutically acceptable carrier or excipient to the patient having the cancer cells in an amount effective to stop or repress the proliferation of the cancer cells. In certain examples, the biomarker(s) may include one or more glycoprotein biomarkers. The method may further comprise sampling the cancer cells from the patient (e.g., by performing a biopsy on a tumor in the patient).

In various embodiments, the drugs may be administered orally, intravenously, or by chemotherapy infusion. For example, the effective drug may be administered orally via a pill or a liquid formulation comprising a dose of the effective drug in an amount effective to stop proliferation of the cancer cells, in a pharmaceutically acceptable carrier or excipient. The effective drug may be administered intravenously or by chemotherapy infusion via an IV bag, an IV drip, or a syringe containing a dose of effective drug in an amount effective to stop proliferation of the cancer cells, in a pharmaceutically acceptable aqueous carrier or excipient. Additionally, the method may further include administering a cancer therapy selected from radiation therapy, surgery, and a combination thereof to the patient.

An Exemplary System Configured to Predict Drugs Effective to Stop or Repress Proliferation or Growth of Cancer Cells

In yet a further aspect, the present invention concerns a system configured to predict effectiveness of one or more drugs to stop or repress proliferation of cancer cells. The system generally comprises a memory storing (i) a first dataset including expression levels of a plurality of proteins or glycoproteins in the plurality of the cancer cell lines, and (ii) a second dataset including an effectiveness of each of a plurality of drugs to stop or repress proliferation of the cancer cell lines, and a computer configured to statistically analyze the first and second datasets to (i) identify and/or select at least one protein or glycoprotein biomarker for each of the cancer cell lines and (ii) correlate or associate at least one of the drugs that effectively stops or represses proliferation of the cancer cells in each of the cancer cell lines with the biomarker(s) for each of the cancer cell lines.

The computer may be configured to statistically analyze the first and second datasets using lasso regression, and optionally, “leave-one-out” analysis. In addition, the first dataset may include expression levels of glycoproteins, and the biomarker may include a glycoprotein biomarker. The system may further include a third dataset that includes expression levels of a plurality of proteins in the same or different plurality of cancer cell lines, in which case the biomarker may include one or more protein biomarkers associated with the drug(s) effective to stop or repress proliferation cancer cells. The first dataset includes expression levels of a plurality of glycoproteins, and at least one biomarker comprises at least one glycoprotein biomarker. In the various embodiments, the effectiveness of each drug to stop or repress proliferation of the cancer cell line is determined by a response that measures a concentration of the drug(s) that causes 50% reduction in proliferation of cancer cells.

The present system further includes algorithms, computer program(s), computer-readable media and/or software, implementable and/or executable in a general purpose computer or workstation equipped with a conventional digital signal processor, and configured to perform one or more of the methods and/or one or more operations of the hardware (e.g., computer) disclosed herein. Thus, a further aspect of the invention relates to algorithms and/or software that predict effectiveness of one or more drugs to stop or repress proliferation of cancer cells, and/or that implement part or all of any method disclosed herein. For example, the computer program or computer-readable medium generally contains a set of instructions which, when executed by an appropriate processing device (e.g., a signal processing device, such as a microcontroller, microprocessor or DSP device), is configured to perform the above-described method(s), operation(s), and/or algorithm(s).

The computer-readable medium may comprise any tangible medium that can be read by a signal processing device configured to read the medium and execute code stored thereon or therein, such as a DVD, CD-ROM, flash drive or hard disk drive. Such code may comprise object code, source code and/or binary code. The code is generally digital, and is generally configured for processing by a conventional digital data processor (e.g., a microprocessor, microcontroller, or logic circuit such as a programmable gate array, programmable logic circuit/device or application-specific integrated circuit [ASIC]).

Thus, an aspect of the present invention relates to a non-transitory computer-readable medium, comprising a set of instructions encoded thereon adapted to generate graphics that assist in predicting effectiveness of one or more drugs to stop or repress proliferation of cancer cells, the instructions including one or more instructions to statistically analyze (i) a first dataset of expression levels of proteins or glycoproteins in cancer cells (e.g., one or more cancer cell lines) and (ii) a second dataset of responses of the cancer cells (or cancer cell lines) to various drugs to identify at least one biomarker associated with effective repression of the cancer cells using one or more of the drugs. In addition, the set of instructions include one or more instructions to correlate or associate the biomarker(s) with a response of the cancer cells to at least one of the drugs that effectively stops or represses the growth of the cancer cells. The graphics that assist in predicting drug effectiveness are, in turn, generated by conventional graphics hardware and/or software in the present system that show and/or plot graphs and/or create tables the same as or similar to those shown in the present Figures.

### CONCLUSION/SUMMARY

Thus, the present invention provides a method of identifying one or more of a plurality of drugs effective to stop or repress proliferation of cancer cells, and a system to predict effectiveness of one or more of a plurality of drugs to stop or repress proliferation of cancer cells. The method includes statistically analyzing (i) a first dataset of expression levels of a plurality of proteins or glycoproteins in the cancer cells and (ii) a second dataset of responses of the cancer cells to a plurality of drugs to identify one or more biomarkers associated with effective repression of the cancer cells, and correlating or associating at least one of the one or more biomarker with a response of the cells to at least one of the drugs effective to stop or repress the proliferation of the cancer cells. The method advantageously determines and/or predicts drug sensitivity of a wide variety of cancer cells using a small or limited number of biomarkers (e.g., protein and/or glycoprotein biomarkers).

In addition, the present invention provides a method of treating cancer. The method generally includes identifying at least one biomarker (e.g., one or more protein or glycoprotein biomarkers) in cancer cells from a patient, identifying one or more of a plurality of drugs that effectively stop or repress proliferation of the cancer cells from a correlation or association of the biomarker(s) with effectiveness of the drug(s) to stop or repress the proliferation of the cancer cells expressing the at least one protein or glycoprotein biomarker, and administering the one or more of the plurality of drugs in a pharmaceutically acceptable carrier or excipient to the patient having the cancer cells in an amount effective to stop or repress the proliferation of the cancer cells.

Furthermore, the present invention provides a system configured to predict effectiveness of one or more drugs to stop or repress proliferation of cancer cells. The system includes a memory storing (i) a first dataset including expression levels of a plurality of proteins or glycoproteins in the plurality of the cancer cell lines, and (ii) a second dataset including an effectiveness of each of a plurality of drugs to stop or repress proliferation of the cancer cell lines, and a computer configured to statistically analyze the first and second datasets to (i) identify and/or select at least one protein or glycoprotein biomarker for each of the cancer cell lines and (ii) correlate or associate at least one of the drugs that effectively stops or represses proliferation of the cancer cells in each of the cancer cell lines with the biomarker(s) for each of the cancer cell lines.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A method of identifying one or more of a plurality of drugs effective to stop or repress proliferation of cancer cells, comprising:
statistically analyzing (i) a first dataset of expression levels of a plurality of proteins or glycoproteins in said cancer cells and (ii) a second dataset of responses of said cancer cells to a plurality of drugs to identify one or more biomarkers associated with effective repression of said cancer cells; and
correlating or associating at least one of said one or more biomarkers with a response of the cancer cells to at least one of said plurality of drugs effective to stop or repress the proliferation of the cancer cells.

2. A method according to claim 1, wherein said plurality of proteins or glycoproteins comprise glycoproteins.

3. A method according to claim 2, wherein said one or more biomarkers comprise one or more glycoprotein biomarkers.

4. A method according to claim 1, wherein said first and second datasets are statistically analyzed by lasso regression.

5. A method according to claim 1, wherein said cancer cells are selected from the group consisting of breast cancer cells, lung cancer cells, melanoma cells, prostate cancer cells, ovarian cancer cells, bladder cancer cells, endometrial cancer cells, kidney cancer cells, pancreatic cancer cells, colorectal cancer cells, lymphoma cells, CNS cancer cells, thyroid cancer cells, and leukemia cells.

6. A method according to claim 1, wherein said one or more biomarkers consist of one, two or three biomarkers.

7. A method according to claim 1, wherein said drugs effective to stop proliferation of cancer cells comprise (i) inhibitors of epidermal growth factor receptor and/or human epidermal growth factor receptor 2, (ii) agents that target microtubules, (iii) agents that target tubulin, (iv) agents that target nucleic acids, (v) mTOR inhibitors, (vi) PI3′ kinase inhibitors, and/or (vii) CDK inhibitors, and said biomarkers are selected from the group consisting of receptor tyrosine-protein kinase erbB-2 (PO4626), cathepsin B (P07858), cadherin-13 (P55290), bone marrow stromal antigen 2 (Q10589), neprilysin (P08473), large neutral amino acids transporter small subunit 1 (Q01650), integrin alpha-6 (P23229), dipeptidyl peptidase 1 (P53634), collagen alpha-1 (VI) chain (P12109), neutral amino acid transporter B (Q15758), transcobalamin-1 (P20061), sushi domain-containing protein 2 (Q9UGT4), podocalyxin (000592), laminin subunit beta-1 (P07942), dipeptidyl peptidase 1 (P53634), gamma-interferon-inducible lysosomal thiol reductase (P13284), neuroplalstin (Q9Y639), CD44 antigen (P16070), ubiquitin carboxyl-terminal hydrolase 5 (P45974), solute carrier family 2, facilitated glucose transporter membrane 1 (P11166), and alpha-aminoadipic semialdehyde dehydrogenase (P49419), CD276 antigen (Q5ZPR3), cathepsin Z (Q9UBR2), and serpin H1 (P50454); lysosome membrane protein 2 (Q14108), alpha-aminoadipic semialdehyde dehydrogenase (P49419), isochorismatase domain-containing protein 1 (Q96CN7), beta-mannosidase (000462), glucose-6-phosphate 1-dehydrogenase (P11413), ribonuclease UK114 (P52758), tropomyosin alpha-4 chain (P67936), ganglioside GM2 activator (P17900), granulins (P28799), steryl-sulfatase (P08842), insulin-like growth factor-binding protein 7 (Q16270), lysosomal pro-x carboxypeptidase (P42785), receptor tyrosine-protein kinase erbB-2 (PO4626), transmembrane emp24 domain-containing protein 7 (Q9Y3B3), arylsulfatase A (P15289), mucin-1 (P15941), G2/mitotic-specific cyclin-B1 (P14635), G1/S-specific cyclin-E1 (P24864), thioredoxin-dependent peroxide reductase, mitochondrial (P30048), acylaminoacyl-peptidase, putative (ApeH-1; Q97YB2), and importin subunit alpha-1 (P52292).

8. A method according to claim 7, wherein said drug effective to stop proliferation of cancer cells comprises an inhibitor of EGFR and/or HER2 selected from the group consisting of (i) afatinib, and said one or more glycoprotein biomarkers includes one or more of receptor tyrosine-protein kinase erbB-2 (PO4626), cathepsin B (P07858), cadherin-13 (P55290), bone marrow stromal antigen 2 (Q10589), and sushi domain-containing protein 2 (Q9UGT4), (ii) erlotinib, and said one or more glycoprotein biomarkers includes one or more of sushi domain-containing protein 2 (Q9UGT4), neprilysin (P08473), large neutral amino acids transporter small subunit 1 (Q01650), integrin alpha-6 (P23229), dipeptidyl peptidase 1 (P53634), collagen alpha-1 (VI) chain (P12109), and neutral amino acid transporter B (Q15758), (iii) gefitinib, and said one or more glycoprotein biomarkers includes one or more of transcobalamin-1 (P20061), sushi domain-containing protein 2 (Q9UGT4), podocalyxin (000592), large neutral amino acids transporter small subunit 1 (Q01650), laminin subunit beta-1 (P07942), and dipeptidyl peptidase 1 (P53634), and (iv) lapatinib, and said one or more glycoprotein biomarkers includes one or more of receptor tyrosine-protein kinase erbB-2 (PO4626), gamma-interferon-inducible lysosomal thiol reductase (P13284), neuroplalstin (Q9Y639), cathepsin B (P07858), CD44 antigen (P16070), and bone marrow stromal antigen 2 (Q10589).

9. A method according to claim 7, wherein said drug effective to stop proliferation of cancer cells targets tubulin or microtubules and is selected from the group consisting of (i) paclitaxel, and said one or more protein biomarkers includes one or more of ubiquitin carboxyl-terminal hydrolase 5 (P45974), solute carrier family 2, facilitated glucose transporter membrane 1 (P11166), and alpha-aminoadipic semialdehyde dehydrogenase (P49419), and said one or more glycoprotein biomarkers includes one or more of CD276 antigen (Q5ZPR3), cathepsin Z (Q9UBR2), and serpin H1 (P50454), (ii) docetaxel, and said one or more protein biomarkers includes one or more of lysosome membrane protein 2 (Q14108), alpha-aminoadipic semialdehyde dehydrogenase (P49419), and isochorismatase domain-containing protein 1 (Q96CN7), and said one or more glycoprotein biomarker includes one or more of beta-mannosidase (000462), cathepsin Z (Q9UBR2), and serpin H1 (P50454), and (iii) vinorelbine, and said one or more protein biomarkers includes one or more of glucose-6-phosphate 1-dehydrogenase (P11413), ribonuclease UK114 (P52758), and tropomyosin alpha-4 chain (P67936).

10. A method according to claim 7, wherein said drug effective to stop proliferation of cancer cells targets nucleic acids and is selected from the group consisting of gemcitabine, and said one or more glycoprotein biomarkers includes one or more of ganglioside GM2 activator (P17900), granulins (P28799), and steryl-sulfatase (P08842).

11. A method according to claim 7, wherein said drug effective to stop proliferation of cancer cells comprises an inhibitor of mTOR selected from the group consisting of (i) everolimus, and said one or more glycoprotein biomarkers includes one or more of insulin-like growth factor-binding protein 7 (Q16270), lysosomal pro-x carboxypeptidase (P42785), and receptor tyrosine-protein kinase erbB-2 (PO4626), and (ii) temsirolimus, and said one or more glycoprotein biomarkers includes one or more of transmembrane emp24 domain-containing protein 7 (Q9Y3B3), arylsulfatase A (P15289), and receptor tyrosine-protein kinase erbB-2 (PO4626).

12. A method according to claim 7, wherein said drug effective to stop proliferation of cancer cells comprises an inhibitor of PI3′ kinase, and said one or more glycoprotein biomarkers includes one or more of collagen alpha-1 (VI) chain (P12109), large neutral amino acids transporter small subunit 1 (Q01650), mucin-1 (P15941), and receptor tyrosine-protein kinase erbB-2 (PO4626).

13. A method according to claim 12, wherein said inhibitor of PI3′ kinase is BEZ235.

14. A method according to claim 7, wherein said drug effective to stop proliferation of cancer cells comprises an inhibitor of CDK, and said one or more protein biomarkers includes one or more of G2/mitotic-specific cyclin-B1 (P14635), G1/S-specific cyclin-E1 (P24864), thioredoxin-dependent peroxide reductase, mitochondrial (P30048), acylaminoacyl-peptidase, putative (ApeH-1; Q97YB2), and importin subunit alpha-1 (P52292).

15. A method of treating cancer, comprising:
identifying at least one protein or glycoprotein biomarker in cancer cells from a patient;
identifying one or more of a plurality of drugs that effectively stop or repress proliferation of said cancer cells from a correlation or association of said at least one protein or glycoprotein biomarker with effectiveness of said one or more drugs to stop or repress said proliferation of cancer cell lines expressing said at least one protein or glycoprotein biomarker; and
administering said one or more of said plurality of drugs in a pharmaceutically acceptable carrier or excipient to said patient having said cancer cells in an amount effective to stop or repress said proliferation of said cancer cells.

16. A method according to claim 15, wherein said at least one biomarker comprises a glycoprotein biomarker.

17. A method according to claim 15, wherein said one or more of said plurality of drugs is administered orally, intravenously, or by chemotherapy infusion.

18. A system configured to predict effectiveness of one or more of a plurality of drugs to stop or repress proliferation of cancer cells, comprising:
a memory storing (i) a first dataset including expression levels of a plurality of proteins or glycoproteins in said plurality of said cancer cell lines, and (ii) a second dataset including an effectiveness of each of said plurality of drugs to stop or repress proliferation of said cancer cell lines; and
a computer configured to statistically analyze said first and second datasets to (i) identify and/or select at least one biomarker for each of said cancer cell lines and (ii) correlate or associate at least one of said plurality of drugs that effectively stops or represses proliferation of said cancer cells in each of said cancer cell lines with said at least one biomarker for each of said cancer cell lines.

19. The system of claim 18, wherein said computer is configured to statistically analyze said first and second datasets using lasso regression.

20. The system according to claim 18, wherein said first dataset includes expression levels of a plurality of glycoproteins, and said at least one biomarker comprises at least one glycoprotein biomarker.
